(19) United States
(12) Patent Application Publication
(10) Pub. No.: US 2025/0277076 A1
(43) Pub. Date: Sep. 4, 2025

(54) MUCOADHESIVE HYDROPHILIC COPOLYMERS AND USES THEREOF

(71) Applicant: Alcon Inc., Fribourg (CH)

(72) Inventors: Frank Chang, Cumming, GA (US); Ye Hong, Flower Mound, TX (US); Troy Vernon Holland, Suwanee, GA (US); Adam K. Sniady, Lilburn, GA (US); Yuyang Ji, Peachtree Corners, GA (US)

(21) Appl. No.: 19/068,465

(22) Filed: Mar. 3, 2025

Related U.S. Application Data
(60) Provisional application No. 63/560,858, filed on Mar. 4, 2024.
(60) Provisional application No. 63/560,860, filed on Mar. 4, 2024.

(51) Int. Cl. C08F 230/06; A61K 9/00; A61K 47/32

(52) U.S. Cl. CPC C08F 230/06; A61K 9/0048; A61K 47/32

(57) ABSTRACT

The invention is generally related to a class of water-soluble hydrophilic copolymers each of which comprises a small mole percentage of repeating monomeric units of at least one arylborono-containing vinylic monomer having a pKa of from about 6.4 to about 7.8 and a majority amount of repeating monomeric units of at least one hydrophilic vinylic monomer. The water-soluble hydrophilic copolymers can interact strongly and reversibly with membrane-bound mucins in the eye. When being used in an ophthalmic composition, they can function as a mucoadhesive hydrophilic polymer for prolonging the retention of demulcents and other drugs/comfort agents and also as active ingredients/lubricants due to their high hydrophilicity.

This application claims the benefits under 35 USC §119(e) of U.S. provisional application Nos. 63/560,858 and 63/560,860 both filed on 4 Mar. 2024, herein incorporated by reference in their entireties.

The present invention generally relates to a class of mucoadhesive hydrophilic copolymers which can interacts strongly and reversibly with membrane-bound mucins in eyes for prolonging the retention of demulcents and other drugs/comfort agents and can also act as an active ingredients/lubricants and also to an ophthalmic composition comprising a mucoadhesive hydrophilic copolymer of the invention.

### BACKGROUND

Great efforts have been made to develop ophthalmic compositions for topical application, and in particular artificial tear compositions, which comprise compounds that lubricate and protect the ocular surface. In the context of dry eye disorders, artificial tear compositions can prevent symptoms such as pain and discomfort, can prevent bioadhesion and tissue damage induced by friction, and can encourage the natural healing and restoration of previously damaged tissues.

Ophthalmic compositions are typically developed with a target viscosity to ensure that they are comfortable for the user and do not cause undesirable side effects such as blurring. A suitable viscosity can help ensure that an ophthalmic composition used in dry eye disorders will relieve dry eye-associated symptoms and/or treat the underlying disorder. In drug delivery applications, the viscosity of ophthalmic compositions may be chosen to ensure that a pharmaceutical agent carried in the composition remains in the eye desirably for a longer time.

Natural polymers or chemically-modified derivatives thereof, which display thixotropic and viscoelastic properties, are typically used as lubricants and ocular surface protectants in many ophthalmic compositions existed in the prior art. Examples of such polymers include hydroxypropyl methylcellulose, galactomannans such as guar and hydroxypropyl guar, carboxymethylcellulose, hyaluronic acid, sodium alginate, and the likes. The shear thinning and viscoelastic profiles of those polymers play important roles when mixed with the tear film.

U.S. Pat. No. 6,403,609 disclose ophthalmic compositions containing galactomannan polymers and borate for providing a desired viscosity to the ophthalmic compositions. The cross-linking of galactomannan and borate is responsible for the gel-forming behavior of the described ophthalmic compositions.

U.S. Pat. Nos. 8,685,945, 8,846,641, 10,828,320 and 1,137,6275 disclose ophthalmic compositions containing galactomannan polymers, borate, a cis-diol (e.g., sorbitol or propylene glycol). The gelling behavior and rheological characteristics of the formulations after instillation into the eye are controlled via selection of the cis-diol.

The existing artificial tear compositions primarily rely on the uses of polymers having thixotropic and viscoelastic properties (i.e., their advantageous shearing thinning and viscoelastic profiles) and have met with some success. However, problems in the treatment of dry eye nevertheless remain. It is believed that those known thixotropic polymers used in the existing artificial tear compositions may not interact strongly and reversibly with membrane-bound mucins in the eye so that the retention of those polymers in the eye may not sufficiently long. The use of tear substitutes, while temporarily effective, generally requires repeated application over the course of a patient's waking hours. It is not uncommon for a patient to have to apply artificial tear composition ten to twenty times over the course of the day. Such an undertaking is not only cumbersome and time consuming but is also potentially very expensive.

Therefore, there are still needs for new mucoadhesive hydrophilic copolymers which can interacts strongly and reversibly with membrane-bound mucins in eyes for prolonging the retention of demulcents and other drugs/comfort agents and can also act as an active ingredients/lubricants, and for ophthalmic compositions that are used for dry-eye eye drop formulation in which the system provides comfort, moisture enhancing and even extended resident time on corneal surface.

### SUMMARY

In one aspect, the invention provides a water-soluble hydrophilic copolymer, comprising: from about 0.01% to about 7.0% by mole of repeating monomeric units of at least one arylborono-containing vinylic monomer having an arylborono group and a pKa of from about 6.4 to about 7.8; from about 60.0% to 99.9% by mole of repeating monomeric units of at least one hydrophilic vinylic monomer which is free of phosphorylcholine group and has N_{H-bond doner}−N_{H-bond acceptor}≥−1 in which N_{H-bond doner} and N_{H-bond acceptor} are the H-bond doner and H-bond acceptor counts respectively of the hydrophilic vinylic monomer; from 0% to about 35% by mole of repeating monomeric units of at least one phosphorylcholine-containing vinylic monomer; and 0% to about 0.20% by mole of at least one hydrophilic vinylic crosslinker, wherein the water-soluble hydrophilic copolymer has a polymer-mucin-interaction synergy of at least 50%, wherein the polymer-mucin-interaction synergy (designated as PMIS) is calculated according to Eq. (1)

$$PMIS \% = \frac{{µ}_{mix} - [ {µ}_{hc} + {µ}_{mucin} ]}{[ {µ}_{hc} + {µ}_{mucin} ]}$$

in which μ_{hc} is the viscosity of a first copolymer solution comprising 1.0% by weight of the water-soluble hydrophilic copolymer in phosphate-buffered saline, μ_{mucin} is the viscosity of a first mucin solution comprising 0.6% by weight of mucin type III from porcine stomach in phosphate-buffered saline, μₘᵢₓ is the viscosity of a copolymer-mucin solution obtained by mixing a second copolymer solution with a second mucin solution in equal volumes, wherein the second copolymer solution contains 2.0% by weight of the water-soluble hydrophilic copolymer in phosphate-buffered saline and the second mucin solution contains 1.2% by weight of mucin type III from porcine stomach in phosphate-buffered saline.

In another aspect, the invention provides an ophthalmic composition comprising: from about 0.1 w/v % to about 10 w/v % of a water-soluble hydrophilic copolymer of the invention; one or more buffering agents in an amount sufficient to provide the ophthalmic composition with a pH of from about 6.5 to about 8.2 (preferably from about 6.7 to about 8.0, even more preferably from about 6.9 to about 7.8); and optionally one or more additional excipients and/or one or more additional active ingredients.

### DETAILED DESCRIPTION

Unless defined otherwise, all technical and scientific terms used herein have the same meaning as commonly understood by one of ordinary skill in the art to which this invention belongs. Generally, the nomenclature used herein and the laboratory procedures are well known and commonly employed in the art. Conventional methods are used for these procedures, such as those provided in the art and various general references. Where a term is provided in the singular, the inventors also contemplate the plural of that term. The nomenclature used herein and the laboratory procedures described below are those well-known and commonly employed in the art.

“About” as used herein means that a number referred to as “about” comprises the recited number plus or minus 1-10% of that recited number.

“Optional” or “optionally” means that the subsequently described event or circumstance can or cannot occur, and that the description includes instances where the event or circumstance occurs and instances where it does not.

A “vinyl-based copolymer” refers to a copolymer of at least two different vinylic monomers.

A “vinylic monomer” refers to a compound that has one sole ethylenically-unsaturated group.

The term “soluble”, in reference to a compound or material in a solvent, means that the compound or material can be dissolved in the solvent to give a solution with a concentration of at least about 0.1% by weight at room temperature (i.e., from about 20° C. to about 30° C.).

The term “insoluble”, in reference to a compound or material in a solvent, means that the compound or material can be dissolved in the solvent to give a solution with a concentration of less than 0.005% by weight at room temperature (as defined above).

The term “ethylenically unsaturated group” is employed herein in a broad sense and is intended to encompass any groups containing at least one >C═CH₂ group. Exemplary ethylenically unsaturated groups include without limitation (meth)acryloyl

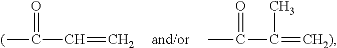

allyl, vinyl (—CH═CH₂), 1-methylethenyl

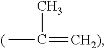

styrenyl, or the likes.

The term “(meth)acrylamide” refers to methacrylamide and/or acrylamide.

The term “(meth)acrylate” refers to methacrylate and/or acrylate.

A “hydrophilic vinylic monomer”, as used herein, refers to a vinylic monomer which can be polymerized to form a homopolymer that is water-soluble or can absorb at least 10 percent by weight of water.

A “hydrophobic vinylic monomer” refers to a vinylic monomer which can be polymerized to form a homopolymer that is insoluble in water and can absorb less than 10 percent by weight of water.

An “acrylic monomer” refers to a vinylic monomer having one sole (meth)acryloyl group.

An “acrylamido monomer” refers to a vinylic monomer having one sole (meth)acrylamido group

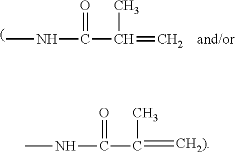

As used in this application, the term “vinylic crosslinker” refers to a compound having at least two ethylenically unsaturated groups. A “vinylic crosslinking agent” refers to a vinylic crosslinker having a molecular weight of about 700 Daltons or less.

As used in this application, the term “polymer” means a material formed by polymerizing/crosslinking one or more monomers or macromers or prepolymers.

As used in this application, the term “molecular weight” of a polymeric material (including monomeric or macromeric materials) refers to the weight-average molecular weight unless otherwise specifically noted or unless testing conditions indicate otherwise.

The term “alkyl” refers to a monovalent radical obtained by removing a hydrogen atom from a linear or branched alkane compound. An alkyl group (radical) forms one bond with one other group in an organic compound.

The term “alkylene divalent radical” or “alkylene diradical” or “alkyl diradical” interchangeably refers to a divalent radical obtained by removing one hydrogen atom from an alkyl. An alkylene divalent group forms two bonds with other groups in an organic compound.

The term “alkyl triradical” refers to a trivalent radical obtained by removing two hydrogen atoms from an alkyl. An alkyl triradical forms three bonds with other groups in an organic compound.

The term “alkoxy” or “alkoxyl” refers to a monovalent radical obtained by removing the hydrogen atom from the hydroxyl group of a linear or branched alkyl alcohol. An alkoxy group (radical) forms one bond with one other group in an organic compound.

In this application, the term “substituted” in reference to an alkyl diradical or an alkyl radical means that the alkyl diradical or the alkyl radical comprises at least one substituent which replaces one hydrogen atom of the alkyl diradical or the alkyl radical and is selected from the group consisting of hydroxy (—OH), carboxy (—COOH), —NH₂, sulfhydryl (—SH), C₁-C₄ alkyl, C₁-C₄ alkoxy, C₁-C₄ alkylthio (alkyl sulfide), C₁-C₄ acylamino, C₁-C₄ alkylamino, di-C₁-C₄ alkylamino, halogen atom (Br or Cl), and combinations thereof.

In this application, an “arylborono-containing vinylic monomer” refers to a vinylic monomer which comprises one sole arylborono group linked to its sole ethylenically unsaturated group through one linkage; an “arylborono-containing acrylic monomer” refers to an acrylic monomer which comprises one sole arylborono group linked to its sole (meth)acryloyl group through one linkage; and an “arylborono-containing acrylamido monomer” refers to a vinylic monomer which comprises one sole arylborono group linked to its sole (meth)acrylamido group through one linkage.

In this application, an “arylborono” group refers to a monovalent radical of

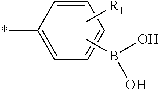

in which R₁ is a monovalent radical (preferably H, NO₂, F, Cl, Br, CF₃, CH₂OH, or CH₂NR⁰R^{o}′ in which R^{o} and R^{o}′ independent of each other are H or C₁-C₄ alkyl). It is understood that where R₁ is CH₂OH, or CH₂NR^{o}R^{o}′, it is at the ortho-position of the boronic acid and can form intramolecular B—O or B—N coordination to lower the pKa of the boronic acid.

In this application, an “phosphorylcholine-containing vinylic monomer” refers to a vinylic monomer which comprises one sole arylborono group linked to its sole ethylenically unsaturated group through one linkage.

As used in this application, the term “phosphorylcholine” refers to a monovalent zwitterionic group of

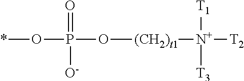

in which t1 is an integer of 1 to 5 and T₁, T₂ and T₃ independently of one another are C₁-C₈ alkyl or C₁-C₈ hydroxyalkyl.

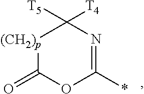

The term “azlactone” refers to a mono-valent radical of in which p is 0 or 1; T₄ and T₅ independently of each other is an alkyl group having 1 to 14 carbon atoms, a cycloalkyl group having 3 to 14 carbon atoms, an aryl group having 5 to 12 ring atoms, an arenyl group having 6 to 26 carbon and 0 to 3 sulfur, nitrogen and/or oxygen atoms, or T₄ and T₅ taken together with the carbon to which they are joined can form a carbocyclic ring containing 5 to 8 ring atoms.

An “initiator” refers to a chemical that can initiate free radical crosslinking and/or polymerizing reaction.

A “mucoadhesive polymer” refers to a polymer capable of bind to a mucus or mucous membrane that adheres to epithelial surfaces (e.g., the gastrointestinal tract, the lung, the eye, etc.), as known to a person skilled in the art. It should point out that mucoadhesive polymers have been widely described in the literature. See, for example, the article entitled “Mucoadhesive Drug Delivery System: A Review” by Dharmendra et al. in Int. J. Pharm. Biol. Arch. 2012, 3(6): 1287-1291 and the article entitled “Polymers in Mucoadhesive Drug-Delivery Systes: A Brief Note” published by Roy et al. in Designed Monomers and Polymers 2009, 12(6): 483-495.

In this application, a “galactomannan polymer” refers to guar or chemically-modified guars. The examples of chemically-modified guars include without limitation hydroxypropyl guar (HP-guar), phosphorylcholine-modified guars (PC-guar) (as described in U.S. Pat. Appl. No. 62/767,5689).

In this application, a “phosphorylcholine-modified galactomannan polymer” refers to a polymer which is a reaction product of a galactomannan polymer with a compound having one reactive acetal group of

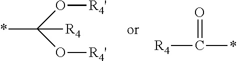

and one phosphorylcholine group and comprises phosphorylcholine groups covalently attached onto the galactomannan polymer each via one 6-membered acetal ring.

In general, the invention provides a class of water-soluble hydrophilic copolymers each of which comprises a small mole percentage (e.g., from about 0.01% to about 7.0% by mole) of repeating monomeric units of at least one arylborono-containing vinylic monomer having an arylborono group and a pKa of from about 6.4 to about 7.8 and a majority amount of repeating monomeric units of at least one hydrophilic vinylic monomer that is free of phosphorylcholine group and has N_{H-bond doner}−N_{H-bond acceptor}≥−1 in which NH-bond doner and NH-bond acceptor are the H-bond doner and H-bond acceptor counts respectively of the hydrophilic vinylic monomer. It has been found that one or more arylborono-containing vinylic monomer having an arylborono group and a pKa of from about 6.4 to about 7.8 can be copolymerized with one or more hydrophilic vinylic monomer that is free of phosphorylcholine group and has N_{H-bond doner}−N_{H-bond acceptor}≥−1 to form water-soluble hydrophilic copolymers that can interacts strongly and reversibly with membrane-bound mucins in the eye. It is believed that the pendant arylborono groups of a water-soluble hydrophilic copolymer can form strong and reversible cyclo boronate ester linkages at a neutral pH with the cis-diol moieties of mucins in the eye to form 3-dimensional polymer network, thereby prolonging the retention of the water-soluble hydrophilic copolymer and demulcents and other drugs/comfort agents in the eye (the tear has a neutral pH). However, due to the hydrophobic nature of an arylborono-containing vinylic hydrophilic, the content of the arylborono-containing vinylic monomer needs to be minimized while the content of one or more hydrophilic vinylic monomers needs to be maximized to ensure that resultant copolymers are water-soluble and have a high hydrophilicity for functioning as an active ingredient/lubricant. It is found that a hydrophilic vinylic monomer that is free of phosphorylcholine group and has N_{H-bond doner}−N_{H-bond acceptor}≥−1 can be used in forming a water-soluble hydrophilic copolymer that can interacts strongly and reversibly with membrane-bound mucins in the eye. It is believed that hydrogen bonds formed between a water-soluble hydrophilic copolymer and mucins in the eye can assist the formation of the 3-dimensional network for prolonging the retention of the water-soluble hydrophilic copolymer and demulcents and other drugs/comfort agents in the eye.

The water-soluble hydrophilic copolymers of the invention are mucoadhesive hydrophilic copolymers and can find particular uses in making ophthalmic compositions in eyes for prolonging the retention of demulcents and other drugs/comfort agents. They can also act as an active ingredients/lubricants due to their high hydrophilicity. When a water-soluble hydrophilic copolymer of the invention comprises repeating monomeric units of at least one phosphorylcholine-containing vinylic monomer, such a water-soluble hydrophilic copolymer may strengthen and stabilize the tear film lipid layer due to the presence of phosphorylcholine groups that have a biomembrane-like structure and a high water-binding (holding) capability.

The invention, in one aspect, provides a water-soluble hydrophilic copolymer, comprising: from about 0.01% to about 7.0% (preferably from about 0.1% to about 6.0%, more preferably from about 0.5% to about 5.0%, even more preferably from about 1.0% to about 4.0%) by mole of repeating monomeric units of at least one arylborono-containing vinylic monomer having an arylborono group and a pKa of from about 6.4 to about 7.8 (preferably from about 6.8 to about 7.6, more preferably from about 6.9 to about 7.5); from about 60.0% to 99.9% (preferably from about 60% to about 94.9%, more preferably from about 65% to about 94%, even more preferably from about 70% to about 93%) by mole of repeating monomeric units of at least one hydrophilic vinylic monomer which is free of phosphorylcholine group and has N_{H-bond doner}−N_{H-bond acceptor}≥−1 in which N_{H-bond doner} and N_{H-bond acceptor} are the H-bond doner and H-bond acceptor counts respectively of the hydrophilic vinylic monomer; from 0% to about 35% (preferably from about 5% to about 30%, more preferably from about 10% to about 25%) by mole of repeating monomeric units of at least one phosphorylcholine-containing vinylic monomer; and from 0% to about 0.20% (preferably from 0% to about 0.15%, more preferably from 0% to about 0.1%) by mole of repeating units of at least one hydrophilic vinylic crosslinker, wherein the water-soluble hydrophilic copolymer has a polymer-mucin-interaction synergy of at least 50%, wherein the polymer-mucin-interaction synergy (designated as PMIS) is calculated according to Eq. (1)

$$PMIS \% = \frac{{µ}_{mix} - [ {µ}_{hc} + {µ}_{mucin} ]}{[ {µ}_{hc} + {µ}_{mucin} ]}$$

in which μ_{hc} is the viscosity of a first copolymer solution comprising 1.0% by weight of the water-soluble hydrophilic copolymer in phosphate-buffered saline, μ_{mucin} is the viscosity of a first mucin solution comprising 0.6% by weight of mucin type III from porcine stomach in phosphate-buffered saline, μₘᵢₓ is the viscosity of a copolymer-mucin solution obtained by mixing a second copolymer solution with a second mucin solution in equal volumes, wherein the second copolymer solution contains 2.0% by weight of the water-soluble hydrophilic copolymer in phosphate-buffered saline and the second mucin solution contains 1.2% by weight of mucin type III from porcine stomach in phosphate-buffered saline.

In accordance with the invention, any suitable arylborono-containing vinylic monomer can be used in the invention so long as it has a pKa of from about 6.4 to about 7.8 (preferably from about 6.8 to about 7.6, more preferably from about 6.9 to about 7.5).

Examples of preferred arylborono-containing vinylic monomers include without limitation 4-(1,6-Dioxo-2,5-diaza-7-oxamyl)phenylboronic acid (pKa˜7.8), 2-dimethylamino-methyl-5-vinylphenylboronic acid (pKa<7.8), 4-(N-allylsulfamoyl)phenylboronic acid (pKa˜7.4), 4-(3-Butenylsulfonyl)phenylboronic acid (pKa˜7.1), 4-acrylamido-3-chlorophenylboronic acid (pKa˜7.5-7.8), 3-(meth)acrylamido-4-nitrophenylboronic acid (pKa˜6.5-6.9), 4-(meth)acrylamido-3-nitrophenylboronic acid (pKa˜6.9), 3-(meth)acrylamido-6-dimethylaminomethylphenylboronic acid (pKa˜7.8), 4-(meth)acrylamido-6-dimethylaminomethylphenylboronic acid, arylborono-containing acrylamido monomers each having a (carbamoylphenyl)boronic acid linked to a (meth)acrylamido group. Preferably, acrylamido monomers each having a (carbamoylphenyl)boronic acid linked to a (meth)acrylamido group are used in preparing a water-soluble hydrophilic copolymer of the invention.

In a preferred embodiment, arylborono-containing acrylamido monomers each having a (carbamoylphenyl)boronic acid linked to a (meth)acrylamido group are represented by formula (I)

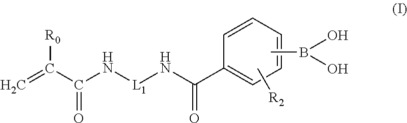

in which R₀ is H or CH₃, R₂ is NO₂, F, Cl, Br, CF₃, CCl₃, or CH₂N(CH₃)₂, and L₁ is a C₂-C₆ alkylene divalent radical or a divalent radical —R₃—X₁—R₄— in which X₁ is an amide linkage of —C(O)NH—, R₃ and R₄ independent of each other are a C₂-C₆ alkylene divalent radical which is optionally substituted with one or more hydroxyl groups, wherein the acrylamido monomer has a pKa of from about 6.4 to about 7.8. Preferably, the boronic acid is at para position.

Where an arylborono-containing acrylamido monomer of formula (I) in which L₁ is a C₂-C₆ alkylene divalent radical can be prepared from the following starting materials: (1) an amino-C₂-C₆-alkyl (meth)acrylamide; and (2) a carboxy-containing phenylboronic acid having a chloro- or nitro-substituent therein, according to a known coupling reaction between carboxylic acid and amine in the presence of a zero-length coupling agent (e.g., 1-ethyl-3-(3-dimethylaminopropyl) carbodiimide (EDC), N,N′-dicyclohexylcarbodiimide (DCC), 1-cylcohexyl-3-(2-morpholinoethyl)carbodiimide, N, N′-diisopropyl carbodiimide (DIC), or mixtures thereof) and an activating agent (e.g., N-hydroxysuccinimide (NHS)) for forming an amide linkage as known in the art (as illustrated in Example 1).

Similarly, one can prepare an arylborono-containing acrylamido monomer of formula (I), in which L₁ is a divalent radical —R₃—X₁—R₄-in which X₁ is an amide linkage of —C(O)NH—, R₃ and R₄ independent of each other are a C₂-C₆ alkylene divalent radical which is optionally substituted with one or more hydroxyl groups, can be prepared from (1) a carboxy-containing (meth)acrylamide or an azlactone-containing vinylic monomer, (2) a carboxy-containing phenylboronic acid having a chloro-or nitro-substituent therein, and (3) an diamine (preferably a mono-Boc-protected diamine), according to the above-described amino-carboxy coupling reaction (to form an amide bond) and ring-opening coupling reaction between azlactone group and amino group —NHR′ (to form an alkylene-diamido linkage of —C(O)NH—CT₄T₅—(CH₂)ₚ—C(O)NR′—).

Examples of preferred carboxy-containing phenylboronic acid having a chloro- or nitro-substituent therein include without limitation 4-carboxy-3-chlorophenylboronic acid, 4-carboxy-2-chlorophenylboronic acid, 4-carboxy-3-nitrophenylboronic acid, 4-carboxy-2-nitrophenylboronic acid, 3-carboxy-4-chlorophenylbornic acid, 3-carboxy-5-chloro-phenylboronic acid, 3-carboxy-2-chlorophenylboronic acid, 3-carboxy-5-nitrophenylboronic acid, 5-carboxy-2-chlorophenylboronic acid, 2-carboxy-4-chlorophenylboronic acid, 2-carboxy-5-chlorophenylboronic acid, and combinations thereof. These preferred carboxy-containing phenylboronic acid compounds can be obtained from commercial sources.

Examples of preferred amino —C₂-C₆-alkyl (meth)acrylamides include without limitation N-(2-aminoethyl) (meth)acrylamide, N-(3-aminopropyl) (meth)acrylamide, N-(2-aminoisopropyl) (meth)acrylamide, N-(4-aminobutyl) (meth)acrylamide, N-(5-aminopentyl) (meth)acrylamide, N-(6-aminohexyl) (meth)acrylamide, and combinations thereof.

Examples of preferred carboxy-containing (meth)acrylamides include without limitation N-(2-carboxypropyl) (meth)acrylamide, N-(3-carboxypropyl) (meth)acrylamide, N-2-acrylamidoglycolic acid, and combinations thereof.

Preferred examples of azlactone-containing vinylic monomers include without limitation 2-vinyl-4,4-dimethyl-1,3-oxazolin-5-one, 2-isopropenyl-4,4-dimethyl-1,3-oxazolin-5-one, 2-vinyl-4-methyl-4-ethyl-1,3-oxazolin-5-one, 2-isopropenyl-4-methyl-4-butyl-1,3-oxazolin-5-one, 2-vinyl-4,4-dibutyl-1,3-oxazolin-5-one, 2-isopropenyl-4-methyl-4-dodecyl-1,3-oxazolin-5-one, 2-isopropenyl-4,4-diphenyl-1,3-oxazolin-5-one, 2-isopropenyl-4,4-pentamethylene-1,3-oxazolin-5-one, 2-isopropenyl-4,4-tetramethylene-1,3-oxazolin-5-one, 2-vinyl-4,4-diethyl-1,3-oxazolin-5-one, 2-vinyl-4-methyl-4-nonyl-1,3-oxazolin-5-one, 2-isopropenyl-4-methyl-4-phenyl-1,3-oxazolin-5-one, 2-isopropenyl-4-methyl-4-benzyl-1,3-oxazolin-5-one, 2-vinyl-4,4-pentamethylene-1,3-oxazolin-5-one, and 2-vinyl-4,4-dimethyl-1,3-oxazolin-6-one (with 2-vinyl-4,4-dimethyl-1,3-oxazolin-5-one (VDMO) and 2-isopropenyl-4,4-dimethyl-1,3-oxazolin-5-one (IPDMO) as most preferred azlactone-containing vinylic monomers).

Examples of preferred diamines include without limitation N,N′-bis(hydroxyethyl)-ethylenediamine, N,N′-dimethylethylenediamine, ethylenediamine, N,N′-dimethyl-1,3-propanediamine, N,N′-diethyl-1,3-propanediamine, propane-1,3-diamine, butane-1,4-diamine, pentane-1,5-diamine, hexamethylenediamine, and combinations thereof.

Examples of such preferred acrylamido monomers of formula (I) include without limitation:

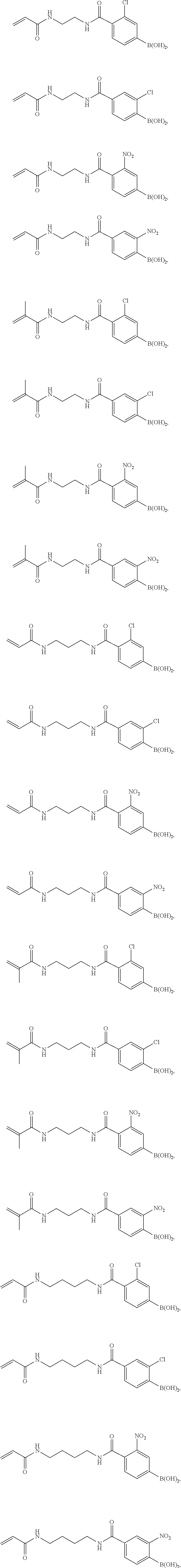

or combinations thereof.

In accordance with the invention, any suitable hydrophilic vinylic monomers can be used in the invention so long as it is free of phosphorylcholine group and has N_{H-bond doner}−N_{H-bond acceptor}≥−1 in which N_{H-bond doner} and N_{H-bond acceptor} are the H-bond doner and H-bond acceptor counts respectively of the hydrophilic vinylic monomer. Preferred examples of such hydrophilic vinylic monomers include without limitation hydrophilic acrylamido monomer (e.g., (meth)acrylamide, N,N-dimethyl (meth)acrylamide, N,N-diethyl (meth)acrylamide, N-ethyl (meth)acrylamide, N-propyl (meth)acrylamide, N-isopropyl (meth)acrylamide, N-2-hydroxylethyl (meth)acrylamide, N,N-bis(hydroxyethyl) (meth)acrylamide, N-3-hydroxypropyl (meth)acrylamide, N-2-hydroxypropyl (meth)acrylamide, N-2-aminoethyl (meth)acrylamide, N-2-methylaminoethyl (meth)acrylamide, N-2-ethylaminoethyl (meth)acrylamide, N-2-dimethylaminoethyl (meth)acrylamide, N-3-aminopropyl (meth)acrylamide, N-3-methylamino-propyl (meth)acrylamide, 2-(meth)acrylamidoglycolic acid, 2-(meth)acrylamidopropionic acid, 3-(meth)acrylamidopropionic acid, 4-(meth)acrylamidobutanoic acid, 5-(meth)acrylamido-pentanoic acid, and combinations thereof), (meth)acrylic acid, ethylacrylic acid, propylacrylic acid, N-vinyl amide monomers (as described below), methylene-containing pyrrolidone monomers (i.e., pyrrolidone derivatives each having a methylene group connected to the pyrrolidone ring at 3-or 5-position) (as described below), N-2-hydroxyethyl vinyl carbamate, and combinations thereof.

Examples of preferred N-vinyl amide monomers include without limitation N-vinylpyrrolidone (aka, N-vinyl-2-pyrrolidone), N-vinyl-3-methyl-2-pyrrolidone, N-vinyl-4-methyl-2-pyrrolidone, N-vinyl-5-methyl-2-pyrrolidone, N-vinyl-6-methyl-2-pyrrolidone, N-vinyl-3-ethyl-2-pyrrolidone, N-vinyl-4,5-dimethyl-2-pyrrolidone, N-vinyl-5,5-dimethyl-2-pyrrolidone, N-vinyl-3,3,5-trimethyl-2-pyrrolidone, N-vinyl piperidone (aka, N-vinyl-2-piperidone), N-vinyl-3-methyl-2-piperidone, N-vinyl-4-methyl-2-piperidone, N-vinyl-5-methyl-2-piperidone, N-vinyl-6-methyl-2-piperidone, N-vinyl-6-ethyl-2-piperidone, N-vinyl-3,5-dimethyl-2-piperidone, N-vinyl-4,4-dimethyl-2-piperidone, N-vinyl caprolactam (aka, N-vinyl-2-caprolactam), N-vinyl-3-methyl-2-caprolactam, N-vinyl-4-methyl-2-caprolactam, N-vinyl-7-methyl-2-caprolactam, N-vinyl-7-ethyl-2-caprolactam, N-vinyl-3,5-dimethyl-2-caprolactam, N-vinyl-4,6-dimethyl-2-caprolactam, N-vinyl-3,5,7-trimethyl-2-caprolactam, N-vinyl-N-methyl acetamide, N-vinyl formamide, N-vinyl acetamide, N-vinyl isopropylamide, N-vinyl-N-ethyl acetamide, N-vinyl-N-ethyl formamide, and mixtures thereof. Preferably, the N-vinyl amide monomer is N-vinylpyrrolidone, N-vinyl-N-methyl acetamide, or combinations thereof.

Examples of preferred methylene-containing (═CH₂) pyrrolidone monomers include without limitations 1-methyl-3-methylene-2-pyrrolidone, 1-ethyl-3-methylene-2-pyrrolidone, 1-methyl-5-methylene-2-pyrrolidone, 1-ethyl-5-methylene-2-pyrrolidone, 5-methyl-3-methylene-2-pyrrolidone, 5-ethyl-3-methylene-2-pyrrolidone, 1-n-propyl-3-methylene-2-pyrrolidone, 1-n-propyl-5-methylene-2-pyrrolidone, 1-isopropyl-3-methylene-2-pyrrolidone, 1-isopropyl-5-methylene-2-pyrrolidone, 1-n-butyl-3-methylene-2-pyrrolidone, 1-tert-butyl-3-methylene-2-pyrrolidone, allyl alcohol, and combinations thereof.

In a preferred embodiment, at least one hydrophilic acrylamido monomer is used in the preparation of a water-soluble hydrophilic copolymer of the invention. Preferably, a hydrophilic acrylamido monomer free of any ionic group is used in the invention. Examples of such preferred non-ionic acrylamido monomers include without limitation (meth)acrylamide, N,N-dimethyl (meth)acrylamide, N-ethyl (meth)acrylamide, N-propyl (meth)acrylamide, N-isopropyl (meth)acrylamide, N-2-hydroxylethyl (meth)acrylamide, N,N-bis(hydroxyethyl) (meth)acrylamide, N-3-hydroxypropyl (meth)acrylamide, N-2-hydroxypropyl (meth)acrylamide, and combinations thereof.

Examples of preferred phosphorylcholine-containing vinylic monomers include without limitation (meth)acryloyloxyethyl phosphorylcholine (aka, MPC, or 2-((meth)acryloyloxy)ethyl-2′-(trimethylammonio)ethylphosphate), (meth)acryloyloxypropyl phosphorylcholine (aka, 3-((meth)acryloyloxy)propyl-2′-(trimethylammonio)ethylphosphate), 4-((meth)acryloyloxy)butyl-2′-(trimethylammonio)ethylphosphate, 2-[(meth)acryloylamino]-ethyl-2′-(trimethylammonio)-ethylphosphate, 3-[(meth)acryloylamino]propyl-2′-(trimethyl-ammonio)ethylphosphate, 4-[(meth)acryloylamino]butyl-2′-(trimethylammonio)ethyl-phosphate, 5-((meth)acryloyloxy)pentyl-2′-(trimethylammonio)ethyl phosphate, 6-((meth)acryloyloxy)hexyl-2′-(trimethylammonio)-ethylphosphate, 2-((meth)acryloyloxy)ethyl-2′-(triethylammonio)ethylphosphate, 2-((meth)acryloyloxy)ethyl-2′-(tripropylammonio)ethyl-phosphate, 2-((meth)acryloyloxy)ethyl-2′-(tributylammonio)ethyl phosphate, 2-((meth)acryloyloxy)propyl-2′-(trimethylammonio)-ethylphosphate, 2-((meth)acryloyloxy)-butyl-2′-(trimethylammonio)ethylphosphate, 2-((meth)acryloyloxy)pentyl-2′-(trimethylammonio)ethylphosphate, 2-((meth)acryloyloxy)hexyl-2′-(trimethylammonio)ethyl phosphate, 2-(vinyloxy)ethyl-2′-(trimethylammonio)ethylphosphate, 2-(allyloxy)ethyl-2′-(trimethylammonio)ethylphosphate, 2-(vinyloxycarbonyl)ethyl-2′-(trimethylammonio)ethyl phosphate, 2-(allyloxycarbonyl)ethyl-2′-(trimethylammonio)-ethylphosphate, 2-(vinylcarbonylamino)ethyl-2′-(trimethylammonio)ethylphosphate, 2-(allyloxycarbonylamino)-ethyl-2′-(trimethylammonio)ethyl phosphate, 2-(butenoyloxy)ethyl-2′-(trimethylammonio)-ethylphosphate, and combinations thereof.

Any suitable hydrophilic vinylic crosslinkers can be used in the invention. Examples of preferred hydrophilic vinylic crosslinkers include without limitation ethyleneglycol di-(meth)acrylate, diethyleneglycol di-(meth)acrylate, triethyleneglycol di-(meth)acrylate, tetraethyleneglycol di-(meth)acrylate, polyethylene glycol di-(meth)acrylate having a number averaged molecular weight of from 200 to 1,000 daltons, glycerol di-(meth)acrylate, glycerol 1,3-diglycerolate di-(meth)acrylate, ethylenebis[oxy(2-hydroxypropane-1,3-diyl)]di-(meth)acrylate, bis[2-(meth)acryloxyethyl]phosphate, diacrylamide (i.e., N-(1-oxo-2-propenyl)-2-propenamide), dimethacrylamide (i.e., N-(1-oxo-2-methyl-2-propenyl)-2-methyl-2-propenamide), N,N-di(meth)acryloyl-N-methylamine, N,N-di(meth)acryloyl-N-ethylamine, N,N′-methylene bis(meth)acrylamide, N,N′-ethylene bis(meth)acrylamide, N,N′-dihydroxyethylene bis(meth)acrylamide, N,N′-propylene bis(meth)acrylamide, N,N′-2-hydroxypropylene bis(meth)acrylamide, N,N′-2,3-dihydroxybutylene bis(meth)acrylamide, 1,3-bis(meth)acrylamide-propane-2-yl dihydrogen phosphate (i.e., N,N′-2-phosphonyloxy-propylene bis(meth)acrylamide), piperazine diacrylamide (or 1,4-bis(meth)acryloyl piperazine), and combinations thereof, more preferably selected from the group consisting of ethyleneglycol di-(meth)acrylate, diethyleneglycol di-(meth)acrylate, triethyleneglycol di-(meth)acrylate, tetraethyleneglycol di-(meth)acrylate, polyethylene glycol di-(meth)acrylate having a number averaged molecular weight of from 200 to 1000 daltons, N,N-di(meth)acryloyl-N-methylamine, N,N-di(meth)acryloyl-N-ethylamine, N,N′-methylene bis(meth)acrylamide, N,N′-ethylene bis(meth)acrylamide, N,N′-dihydroxyethylene bis(meth)acrylamide, N,N′-propylene bis(meth)acrylamide, N,N′-2-hydroxypropylene bis(meth)acrylamide, N,N′-2,3-dihydroxybutylene bis(meth)acrylamide, combinations thereof.

In accordance with the invention, a water-soluble hydrophilic copolymer of the invention can be a linear or branched polymer, so long as it can be dissolved in water.

In accordance with the invention, the mole percentages of each type of repeating monomeric units of a water-soluble hydrophilic copolymer of the invention can be determined based on the mole percentage of each type of vinylic monomers, from which this type of repeating units are derived, in a polymerizable composition for forming a water-soluble hydrophilic copolymer.

In accordance with the invention, a water-soluble hydrophilic copolymer of the invention has a number average molecular weight of from about 10,000 Daltons to about 10,000,000 Daltons, preferably from about 25,000 Daltons to 5,000,000 Daltons, more preferably from about 50,000 Daltons to about 2,000,000 Daltons.

A person skilled in the art knows well how to prepare a water-soluble hydrophilic copolymer of the invention according to any known polymerization technique. For example, it can be obtained by thermal or actinic polymerization of a polymerizable composition comprising all the required polymerizable components, a free-radical initiator (thermal initiator or photoinitiator), and optionally (but preferably) a chain transfer agent. A chain transfer agent (containing at least one thiol group) is used to control the molecular weight of the resultant copolymer.

The polymerizable composition for preparing a copolymer of the invention can a solution in which all necessary component is dissolved in an inert solvent (i.e., should not interfer with the reaction between the reactants in the mixture), such as water, an organic solvent, or mixture thereof, as known to a person skilled in the art.

Examples of suitable solvents include without limitation, water, tetrahydrofuran, tripropylene glycol methyl ether, dipropylene glycol methyl ether, ethylene glycol n-butyl ether, ketones (e.g., acetone, methyl ethyl ketone, etc.), diethylene glycol n-butyl ether, diethylene glycol methyl ether, ethylene glycol phenyl ether, propylene glycol methyl ether, propylene glycol methyl ether acetate, dipropylene glycol methyl ether acetate, propylene glycol n-propyl ether, dipropylene glycol n-propyl ether, tripropylene glycol n-butyl ether, propylene glycol n-butyl ether, dipropylene glycol n-butyl ether, tripropylene glycol n-butyl ether, propylene glycol phenyl ether dipropylene glycol dimetyl ether, polyethylene glycols, polypropylene glycols, ethyl acetate, butyl acetate, amyl acetate, methyl lactate, ethyl lactate, i-propyl lactate, methylene chloride, 2-butanol, 1-propanol, 2-propanol, menthol, cyclohexanol, cyclopentanol and exonorborneol, 2-pentanol, 3-pentanol, 2-hexanol, 3-hexanol, 3-methyl-2-butanol, 2-heptanol, 2-octanol, 2-nonanol, 2-decanol, 3-octanol, norborneol, tert-butanol, tert-amyl, alcohol, 2-methyl-2-pentanol, 2,3-dimethyl-2-butanol, 3-methyl-3-pentanol, 1-methylcyclohexanol, 2-methyl-2-hexanol, 3,7-dimethyl-3-octanol, 1-chloro-2-methyl-2-propanol, 2-methyl-2-heptanol, 2-methyl-2-octanol, 2-2-methyl-2-nonanol, 2-methyl-2-decanol, 3-methyl-3-hexanol, 3-methyl-3-heptanol, 4-methyl-4-heptanol, 3-methyl-3-octanol, 4-methyl-4-octanol, 3-methyl-3-nonanol, 4-methyl-4-nonanol, 3-methyl-3-octanol, 3-ethyl-3-hexanol, 3-methyl-3-heptanol, 4-ethyl-4-heptanol, 4-propyl-4-heptanol, 4-isopropyl-4-heptanol, 2,4-dimethyl-2-pentanol, 1-methylcyclopentanol, 1-ethylcyclopentanol, 1-ethylcyclopentanol, 3-hydroxy-3-methyl-1-butene, 4-hydroxy-4-methyl-1-cyclopentanol, 2-phenyl-2-propanol, 2-methoxy-2-methyl-2-propanol 2,3,4-trimethyl-3-pentanol, 3,7-dimethyl-3-octanol, 2-phenyl-2-butanol, 2-methyl-1-phenyl-2-propanol and 3-ethyl-3-pentanol, 1-ethoxy-2-propanol, 1-methyl-2-propanol, t-amyl alcohol, isopropanol, 1-methyl-2-pyrrolidone, N,N-dimethylpropionamide, dimethyl formamide, dimethyl acetamide, dimethyl propionamide, N-methyl pyrrolidinone, and mixtures thereof.

The copolymerization of a polymerizable composition for preparing a copolymer of the invention may be induced photochemically or preferably thermally. Suitable thermal polymerization initiators are known to the skilled artisan and comprise, for example peroxides, hydroperoxides, azo-bis (alkyl-or cycloalkylnitriles), persulfates, percarbonates or mixtures thereof. Examples are benzoylperoxide, tert-butyl peroxide, di-tert.-butyl-diperoxy-phthalate, tert.-butyl hydroperoxide, azo-bis(isobutyronitrile) (AIBN), 1,1-azodiisobutyramidine, 1,1′-azo-bis(1-cyclohexanecarbonitrile), 2,2′-azo-bis(2,4-dimethyl-valeronitrile) and the like. The polymerization is carried out conveniently in an above-mentioned solvent at elevated temperature, for example at a temperature of from 25 to 100° C. and preferably 40 to 80° C. The reaction time may vary within wide limits, but is conveniently, for example, from 1 to 24 hours or preferably from 2 to 12 hours. It is advantageous to previously degas the components and solvents used in the polymerization reaction and to carry out said copolymerization reaction under an inert atmosphere, for example under a nitrogen or argon atmosphere. Copolymerization can yield optical clear well-defined copolymers which may be worked up in conventional manner using for example extraction, precipitation, ultrafiltration and the like techniques.

A water-soluble hydrophilic copolymer of the invention can find particular use in developing ophthalmic compositions for topical application, and in particular artificial tear compositions, which comprise compounds that lubricate and protect the ocular surface. In the context of dry eye disorders, artificial tear compositions can prevent symptoms such as pain and discomfort, can prevent bioadhesion and tissue damage induced by friction, and can encourage the natural healing and restoration of previously damaged tissues.

In another aspect, the invention provides an ophthalmic composition comprising: from about 0.1 w/v % to about 10 w/V % of a water-soluble hydrophilic copolymer of the invention; one or more buffering agents in an amount sufficient to provide the ophthalmic composition with a pH of from about 6.5 to about 8.2 (preferably from about 6.7 to about 8.0, even more preferably from about 6.9 to about 7.8); and optionally one or more additional excipients and/or one or more additional active ingredients.

Any known, physiologically compatible buffering agents can be used. Suitable buffering agents include, but are not limited to, phosphates (consisting essentially of a mixture of a monobasic dihydrogen phosphate, e.g., NaH₂PO₄, KH₂PO₄, and a dibasic monohydrogen phosphate, e.g., Na₂HPO₄, K₂HPO₄), acetates, borates, amino alcohols, and mixtures thereof.

Examples of borates include without sodium borate (borax), potassium borate, calcium borate, magnesium borate, manganese borate, and other such borate salts. As used herein, the term “borate” refers to all pharmaceutically suitable forms of borates. Borates are common excipients in ophthalmic formulations due to good buffering capacity at physiological pH and well known safety and compatibility with a wide range of drugs and preservatives. Borates also have inherent bacteriostatic and fungistatic properties, and therefore aid in the preservation of the compositions.

Examples of amino alcohols include without limitation 2-amino-2-methyl-1-propanol (AMP), 2-dimethylamino-methyl-1-propanol (DMAMP), 2-amino-2-ethyl-1,3-propanediol (AEPD), 2-amino-2-methyl-1,3-propanediol (AMPD), 2-amino-1-butanol (AB), 1,3 bis(tris[hydroxymethyl]methylamino)propane (bis-TRIS-propane), or combinations thereof.

Any of a variety of excipients may be used in formulations of the present invention. Excipients commonly used in pharmaceutical formulations include without limitation demulcents, tonicity agents, preservatives, chelating agents, surfactants, solubilizing agents, stabilizing agents, comfort-enhancing agents, polymers, emollients, pH-adjusting agents, lubricants, water, mixtures of water and water-miscible solvents (such as C₁-C₇ alkanols), vegetable oils or mineral oils comprising from 0.5 to 5% non-toxic water-soluble polymers, natural products (such as alginates, pectins, tragacanth, karayagum, Xanthan gum, carrageenin, agar and acacia), starch derivatives (such as starch acetate and hydroxypropyl Starch), synthetic products (such as polyvinyl alcohol, polyvinylpyrrolidone, polyvinyl methyl ether, polyethylene oxide, preferably cross-linked polyacrylic acid, and mixtures of those products), and combinations thereof.

Demulcents used with embodiments of the present invention include, but are not limited to, glycerin, polyvinyl pyrrolidone, polyethylene oxide, polyethylene glycol, polyethyleoxide-polybutyleneoxide block copolymer, polyethyleneoxide-polypropyleneoxide block copolymer, propylene glycol, polyacrylic acid, and combinations thereof. Particularly preferred demulcents are propylene glycol and polyethylene glycol 400.

Suitable tonicity-adjusting agents include without limitation sodium chloride, potassium chloride, glycerol, propylene glycol, polyols, mannitol, sorbitol, xylitol, propylene glycol, polyethylene glycol having a number average molecular weigh of from 200 to 800 daltons, and mixtures thereof.

An ophthalmic composition of the invention is preferably isotonic, or slightly hypotonic in order to combat any hypertonicity of tears caused by evaporation and/or disease. This may require a tonicity-adjusting agent to bring the osmolality of the formulation to a level at or near 210-320 milliosmoles per kilogram (mOsm/kg). The ophthalmic composition of the present invention generally has an osmolality in the range of 220-320 mOsm/kg, and preferably has an osmolality in the range of 235-300 mOsm/kg.

Examples of preservatives include p-hydroxybenzoic acid ester, sodium perborate, sodium chlorite, alcohols such as chlorobutanol, benzyl alcohol or phenyl ethanol, guanidine derivatives such as polyhexamethylene biguanide, polyquaternium-1 (aka, POLYQUAD® or ONAMERM®), or sorbic acid. In certain embodiments, the composition may be self-preserved so that no preservation agent is required.

Suitable surfactants include, but are not limited to, ionic and nonionic surfactants, though nonionic surfactants are preferred, RLM 100, POE 20 cetylstearyl ethers such as Procol® CS20 and poloxamers such as Pluronic® F68, and block copolymers such as poly(oxyethylene)-poly(oxybutylene) compounds set forth in U.S. Pat. Appl. Pub. No. 2008/0138310.

In a preferred embodiment, an ophthalmic composition of the invention further comprises at least one mucoadhesive polymer selected from the group consisting of hydroxypropyl methylcellulose, galactomannans such as guar and hydroxypropyl guar, carboxymethylcellulose, hyaluronic acid, sodium alginate, polyvinyl alcohol, and combinations thereof;

In accordance with the invention, any linear or branched mucoadhesive polymer can be used in the invention, so long as it has reactive moieties selected from the group consisting of 1,2-diol moieties, 1,3-diol moieties, α-hydroxycarboxylic acid moieties, β-hydroxycarboxylic acid moieties, and combinations thereof.

As used in this application, a galactomannan polymer refers to a galactomannan (e.g., guar) and/or a chemically modified galactomannan.

A galactomannan, as known to a person skilled in the art, is a polysaccharide consisting of a mannose backbone with galactose side groups (more specifically, a (1-4)-linked beta-D-mannopyranose backbone with branch points from their 6-positions linked to alpha-D-galactose, (i.e. 1-6-linked alpha-D-galactopyranose). The ratio of D-galactose to D-mannose in galactomannan can vary, but generally will be from about 1:2 to 1:4. Galactomannans having a D-galactose: D-mannose ratio of about 1:2 are most preferred. Preferred galactomannan is guar.

Galactomannans may be obtained from numerous sources. Such sources include guar gum, locust bean gum and tara gum, as further described below.

Guar gum is the ground endosperm of Cyamopisis tetragonolobus (L.) Taub. The water soluble fraction (85%) is called “guaran” (molecular weight of 220,000), which consists of linear chains of (1-4)-β-D mannopyranosyl units with α-D-galactopyranosyl units attached by (1-6) linkages. The ratio of D-galactose to D-mannose in guaran is about 1:2. The gum has been cultivated in Asia for centuries and is primarily used in food and personal care products for its thickening property. It has five to eight times the thickening power of starch. Guar gum may be obtained, for example, from Rhone-Polulenc (Cranbury, N.J.), Hercules, Inc. (Wilmington, Del.) and TIC Gum, Inc. (Belcamp, Md.).

Locust bean gum or carob bean gum is the refined endosperm of the seed of the carob tree, ceratonia siliqua. The ratio of galactose to mannose for this type of gum is about 1:4. Cultivation of the carob tree is old and well known in the art. This type of gum is commercially available and may be obtained from TIC Gum, Inc. (Bekamp, Md.) and Rhone-Polulenc (Cranbury, N.J.).

Tara gum is derived from the refined seed gum of the tara tree. The ratio of galactose to mannose is about 1:3. Tara gum is not produced in the United States commercially, but the gum may be obtained from various sources outside the United States.

A chemically-modified galactomannan is a derivative of a galactomannan in which some (but not all) of hydrogen atoms of the hydroxyl groups are substituted with an organic group. Examples of preferred chemically-modified glactomannans include without limitation hydroxyethyl-substituted galactomannan (e.g., hydroxyethyl guar), hydroxypropyl galactomannan (e.g., hydroxypropyl guar), C₁-C₃ alkyl galactomannan (e.g., methyl guar, ethyl guar, propyl guar), carboxymethyl galactomannan (e.g., carboxymethyl guar), carboxymethylhydroxypropyl galactomannan (e.g., carboxymethylhydroxypropyl guar), hydroxypropyltrimonium chloride galactomannan (e.g., hydroxypropyltrimonium chloride guar), a phosphorylcholine-modified (PC-modified) galactomannan as described in a copending U.S. Pat. Appl. No. (62/767,568), and combinations thereof. Preferred chemically-modified glactomannans are hydroxypropyl guar, phosphorylcholine-modified gaur, or combinations thereof.

Hydroxyethyl guar, hydroxypropyl guar, methyl guar, ethyl guar, propyl guar, carboxymethyl guar, carboxymethylhydroxypropyl guar, and hydroxypropyltrimonium chloride guar are well known and are commercially available. For example, modified galactomannans of various degree of substitution are commercially available from Rhone-Poulenc (Cranbury, N.J.).

In accordance with the invention, the at least one mucoadhesive polymer is present in an amount of from about 0.05 to about 5 w/v %, preferably from about 0.5 to about 2.0 w/v %, more preferably from about 0.2 to about 1.5 w/v %, and most preferably from about 0.25 to about 1.0 w/v %.

Where an ophthalmic composition of the invention comprises at least one mucoadhesive polymer, it preferably comprises at least one cis-diol compound at a concentration that inhibits cross-linking of the mucoadhesive polymer and the water-soluble hydrophilic copolymer. Once instilled in the eye, the cis-diol compound is diluted by the natural tear film allowing a gradual increase in the cross-linking of the mucoadhesive polymer and water-soluble hydrophilic copolymer and a corresponding gradual increase in viscosity and elasticity. This gradual increase in viscosity, cross-linking, and elasticity allows for effective spreading and less blurring upon contact, yet provides long lasting lubrication and corneal surface protection. A cis-diol compound is any compound that comprise hydroxyl groups attached to adjacent carbon atoms. Exemplary cis-diol compounds include without limitation hydrophilic carbohydrates (e.g., sorbitol, mannitol), propylene glycol, glycerol, and combinations thereof. Preferred cis-diol compounds of the present invention include propylene glycol, sorbitol, mannitol and combinations thereof. The cis-diol compounds are present at concentrations of about 0.5 to 5.0 w/v %, preferably about 0.5 to 2.0 w/v % in the ophthalmic compositions of the present invention.

An ophthalmic composition of the invention may be used to administer pharmaceutically active compounds.

Such compounds include without limitation anesthetic drugs, glaucoma therapeutics, pain relievers, anti-hypertensive agents, neuro-protective agents, muco-secretagogue, angiostatic, anti-angiogenesis agents, growth factors, immunosuppressant agents, anti-inflammatory agents, anti-allergy medications, anti-microbials, anti-viral agents, anti-muscarinic agents for myopia treatment, dry eye therapeutics such as PDE4 inhibitors, dopaminergic antagonists, proteins, and the combinations thereof.

Examples of glaucoma therapeutics (or anti-glaucoma agent) include without limitation betaxolol, timolol, pilocarpine, levobetaxolol, apraclonidine, brimonidine, carbonic anhydrase inhibitors (e.g., brinzolamide and dorzolamide), and prostaglandins (e.g., travoprost, bimatoprost, and latanoprost).

Examples of anti-infective agents include without limitation ciprofloxacin, moxifloxacin, and tobramycin.

Examples of anti-inflammatory agents include non-steroidal and steroidal anti-inflammatory agents, such as triamcinolone actinide, naproxen, suprofen, diclofenac, ketorolac, nepafenac, rimexolone, tetrahydrocortisol, and dexamethasone.

Examples of antihypertensive agents include without limitation para-amino clonidine (apraclonidine).

Examples of growth factors include without limitation epidermal growth factor (EGF) and vascular endothelial growth factor (VEGF).

Examples of anti-allergy agents include without limitation olopatadine, epinastine, ketotifen, emedastine, cromolyn.

Examples of antiviral agents include without limitation ganciclovir and valganciclovir. Examples of anti-muscarinic agents include without limitation atropine, pirenzepine, and derivatives thereof.

Anti-angiogenesis agents include anecortave acetate (RETAANE®) and receptor tyrosine kinase inhibitors (RTKi).

Local anesthetic drugs can generally be divided into two categories based on chemical structure: “amides” and “esters.” See Ophthalmic Drug Facts '99, Facts and Comparisons, St. Louis, Mo. (1999), Ch.3. Examples of suitable anesthetic drugs include proparacaine, lidocaine, cocaine, oxybuprocaine, benoxinate, butacaine, mepivacaine, etidocaine, dibucaine, bupivacaine, levobupivacaine, tetracaine and procaine. Most preferred are levobupivacaine, proparacaine and tetracaine.

The ophthalmic compositions of the invention can be particularly useful for delivery therapeutic agents that relieve symptoms of dry eye conditions, cooling agents, antioxidants (omega-3 and omega-6 fatty acids), nutraceuticals (e.g., vitamin A, vitamin D, vitamin E, tocopherols, vitamin K, beta-carotene), and other bioactives for ophthalmic uses. Generally, amounts of therapeutic agent, when used, can be quite variable depending upon the agent or agents used. As such, the concentration of therapeutic agent can be at least about 0.005 w/v %, more typically at least about 0.01 w/v % and even more typically at least about 0.1 w/v %, but typically no greater than about 10 w/v %, more typically no greater than about 4.0 w/v %, still more typically no greater than about 2.0 w/v %.

In a preferred embodiment, an ophthalmic composition of the invention is an aqueous solution. An “aqueous solution” refers to a solution comprising at least 60% by weight, preferably at least 75% by weight, more preferably at least 90% by weight of water.

In another preferred embodiment, an ophthalmic composition of the invention is an emulsion that comprises a water-soluble hydrophilic copolymer of the invention and at least one ophthalmic oil dispersed throughout the continuous water or aqueous phase as small droplets that are substantially distinct and separate. It should be understood that, as used herein, the phase distinct and separate means that, at any given point in time, the droplets are distinct and separate. However, the droplets of the emulsion can combine and separate over time to maintain an average droplet size or diameter. The droplets of the emulsion of the present invention typically have an average or mean diameter no greater than about 1500 nanometers (nm), more typically no greater than about 1000 nm and still more typically no greater than about 600 nm. These droplets also typically have an average or mean diameter that is typically at least 2 nm, more typically at least 10 nm and still more typically at least 100 nm.

Particle or droplet size analyzers may be used to determine emulsion oil droplet size. For example, a Microtrac S3500 Particle Size Analyzer (Software Version 10.3.1) is a tri-laser particle size analyzer that can be used to measure emulsion oil droplet size. That particular analyzer measures laser light diffracted (scattered) from particles (e.g., droplets) in a flowing stream. The intensity and direction of the scattered light is measured by two optical detectors. Mathematical analysis of the diffraction pattern by the software generates a volume distribution of droplet size. The droplet diameter corresponding to 90% of the cumulative undersize distribution by volume is used.

Examples of ophthalmic oils include without limitation any of numerous mineral oils, vegetable oil, synthetic substances, and/or animal and vegetable fats or any combination of oils. The oil can be soluble in various organic solvents such as ether but not in water. The oil phase can comprise, if desired, monoglycerides, diglycerides, triglycerides, glycolipids, glyceroglycolipids, sphingolipids, sphingo-glycolipids, fatty alcohols, hydrocarbons having a C₁₂-C₂₈ chain in length, wax esters, fatty acids, mineral oils, and silicone oils. Mineral oil is particularly preferred. A silicone oil may also be used. The oil phase can additionally include a waxy hydrocarbon, such as paraffin waxes, hydrogenated castor oil, Synchrowax HRC, Carnauba, beeswax, modified beeswaxes, microcrystalline waxes, and polyethylene waxes. The oil is typically at least 0.01 w/v %, more typically at least 0.1 w/v % and even more typically 0.8 w/v % of the emulsion. The oil is also typically no greater than about 20 w/v %, more typically no greater than about 5 w/v % and even more typically no greater than about 3 or even 1.5 w/v % of the emulsion.

The emulsion will also typically include a hydrophilic surfactant (high HLB) and a hydrophobic (low HLB) surfactant. The emulsions of the present invention are most desirably used for dry eye therapeutics. However, without limitation, it is also contemplated that the emulsions may be used for drug delivery, vitamin delivery, botanical delivery, contact lens wetting and contact lens lubrication.

The emulsion of the present invention also typically incorporates two or more surfactants, which act as emulsifiers aiding in the emulsification of the emulsion. Typically, these surfactants are non-ionic. The concentration of emulsifying surfactant in the emulsion is often selected in the range of from 0.1 to 10% w/v, and in many instances from 0.5 to 5% w/v. It is preferred to select at least one emulsifier/surfactant which is hydrophilic and has an HLB value of at least 8 and often at least 10 (e.g., 10 to 18). It is further preferred to select at least one emulsifier/surfactant which is hydrophobic and has an HLB value of below 8 and particularly from 1 to 6. By employing the two surfactants/emulsifiers together in appropriate ratios, it is readily feasible to attain a weighted average HLB value that promotes the formation of an emulsion. For most emulsions according to the present invention, the average HLB value is chosen in the range of about 6 to 12, and for many from 7 to 11. For example, the HLB values for exemplary surfactants and mineral oil are as follows: hydrophobic surfactant (2.1), hydrophilic surfactant (16.9) and mineral oil (10.5).

The hydrophilic surfactant is typically present in the emulsion in an amount that is at least about 0.01 w/v %, more typically at least about 0.08 w/v % and even more typically at least about 0.14 w/v %. The hydrophilic surfactant is typically present in the emulsion in an amount that is no greater than about 1.5 w/v %, more typically no greater than about 0.8 w/v % and even more typically no greater than about 0.44 w/v %.

The hydrophilic surfactant can be a fatty acid, an ester, an ether, an acid or any combination thereof. The hydrophilic surfactant may be ionic or non-ionic, but is preferably non-ionic. Many suitable surfactants/emulsifiers are nonionic ester or ether emulsifiers comprising a polyoxyalkylene moiety, especially a polyoxyethylene moiety, often containing from about 2 to 80, and especially 5 to 60 oxyethylene units, and/or contain a polyhydroxy compound such as glycerol or sorbitol or other alditols as hydrophilic moiety. The hydrophilic moiety can contain polyoxypropylene. The emulsifiers additionally contain a hydrophobic alkyl, alkenyl or aralkyl moiety, normally containing from about 8 to 50 carbons and particularly from 10 to 30 carbons. Examples of hydrophilic surfactants/emulsifiers include ceteareth-10 to -25, ceteth-10-25, steareth-10-25, and PEG-15-25 stearate or distearate. Other suitable examples include C₁₀-C₂₀ fatty acid mono, di or tri-glycerides. Further examples include C₁₈-C₂₂ fatty alcohol ethers of polyethylene oxides (8 to 12 EO). One particularly preferred hydrophilic surfactant is polyoxyethylene-40-stearate, which is sold under the tradename MYRJ-52, which is commercially available from Nikko Chemicals.

The hydrophobic surfactant is typically present in the emulsion in an amount that is at least about 0.01 w/v %, more typically at least about 0.11 w/v % and even more typically at least about 0.16 w/v %. The hydrophobic surfactant is typically present in the emulsion in an amount that is no greater than about 10.0 w/v %, more typically no greater than about 2.0 w/v % and even more typically no greater than about 0.62 w/v %.

The hydrophobic surfactant can be a fatty acid, an ester, an ether, an acid or any combination thereof. The hydrophobic surfactant may be ionic or non-ionic, but is preferably non-ionic. The hydrophobic surfactant will typically include a hydrophobic moiety. The hydrophobic moiety can be either linear or branched and is often saturated, though it can be unsaturated, and is optionally fluorinated. The hydrophobic moiety can comprise a mixture of chain lengths, for example, those deriving from tallow, lard, palm oil sunflower seed oil or soya bean oil. Such non-ionic surfactants can also be derived from a polyhydroxy compound such as glycerol or sorbitol or other alditols. Examples of hydrophobic surfactants include, without limitation, sorbitan fatty acid esters such as sorbitan monoleate, sorbitan monostearate, sorbitan monolaurate, sorbitan monopalmitate, sorbitan monoisostearate, sorbitan trioleate, sorbitan tristearate, sorbitan sesquioleate, sorbitan sesquistearate, combinations thereof or the like. One particularly preferred hydrophobic surfactant is a sorbitan tristearate sold under the tradename SPAN-65, which is commercially available from Croda Worldwide.

The emulsion of the present invention may be formed using a variety of combining and mixing protocol and techniques known to those skilled in the art. According to one preferred embodiment, however, the ingredients are mixed and combined according to a specific protocol. In such protocol, multiple admixtures are formed and those admixtures are combined to form the emulsion. The first admixture is formed by mixing the oil and the surfactants at an elevated temperature to form an oil phase admixture. The second admixture is formed mixing the anionic phospholipid into purified water at an elevated temperature to form a water phase admixture. Thereafter, the oil phase admixture and the water phase admixture are mixed at an elevated temperature and subsequently homogenized using a homogenizer to form an initial emulsion. A third admixture is formed by mixing the galactomannan polymer with water and adjusting pH as needed to form a galactomannan polymer slurry. The galactomannan polymer slurry is then mixed with initial emulsion and form a polymer enhanced emulsion. A fourth admixture is formed by mixing any combination of the following to form a salt solution: borate, polyol, preservative and any other ingredients. The salt solution and the enhanced emulsion are then mixed followed by the addition of a sufficient quantity (Q.S.) of water and pH adjustment.

In accordance with the invention, a composition of the present invention is administered once a day. However, the compositions may also be formulated for administration at any frequency of administration, including once a week, once every 5 days, once every 3 days, once every 2 days, twice a day, three times a day, four times a day, five times a day, six times a day, eight times a day, every hour, or greater frequency. Such dosing frequency is also maintained for a varying duration of time depending on the therapeutic regimen. The duration of a particular therapeutic regimen may vary from one-time dosing to a regimen that extends for months or years. One of ordinary skill in the art would be familiar with determining a therapeutic regimen for a specific indication.

Although various embodiments of the invention have been described using specific terms, devices, and methods, such description is for illustrative purposes only. The words used are words of description rather than of limitation. It is to be understood that changes and variations may be made by those skilled in the art without departing from the spirit or scope of the present invention, which is set forth in the following claims. In addition, it should be understood that aspects of the various embodiments may be interchanged either in whole or in part or can be combined in any manner and/or used together, as illustrated below:

The previous disclosure will enable one having ordinary skill in the art to practice the invention. Various modifications, variations, and combinations can be made to the various embodiment described herein. In order to better enable the reader to understand specific embodiments and the advantages thereof, reference to the following examples is suggested. It is intended that the specification and examples be considered as exemplary.

### Chemicals

The following abbreviations are used in the following examples: CPBA represents (4-(2-acrylamidoethyl)carbamoyl)-3-chlorophenyl) boronic acid; AA represents acrylic acid; APBA represents 3-(acrylamido)phenylboronic acid; MBA represents N,N-methylenebis(acrylamide); MPC represent 2-methacryloyloxyethyl phosphorylcholine; DMA represents N,N-dimethylacrylamide; HEAm represents N-(2-hydroxyethyl)acrylamide; NHS represents N-hydroxysuccinimide; DIC represents diisopropylcarbodiimide; EtOAc represents ethyl acetate; DMF represents dimethylformamide; DIPEA represents N,N-diisopropylethylamine; DMSO represents dimethyl sulfoxide; MeOH represent methanol; mmole represents millimole; TLC represent thin layer chromatograph; CHCl₃ represents chloroform; NMR represents nuclear magnetic resonance; Hz represents hertz; MHz represents megahertz; VAZO 56 represents 2,2′-azobis(2-methylpropionamidine) dihydrochloride; PBS represents a phosphate-buffered saline which has a pH of 7.2±0.2 at 25° C. and contains about 0.044 wt. % NaH₂PO₄·H₂O, about 0.388 wt. % Na₂HPO₄·2H₂O, and about 0.79 wt. % NaCI; DI water represents deionized water; wt. % (% by weight) represents weight percent; w/v % represents a weight/volume percentage concentration (i.e., grams of a solute per 100 mL of solution); min represents minute; MW represents molecular weight; UF represents ultrafiltration; cP represents centipoise; meq represents milliequivalent; MUC2 represent mucin (type III) from porcine stomach (from Sigma-Aldrich, catalogue number M1778); DPBS represents Dulbecco's phosphate-buffered saline.

### Example 1

A (4-(2-Acrylamidoethyl)carbamoyl)-3-chlorophenyl)boronic acid (CPBA) is prepared according to the procedure shown in Scheme 1.

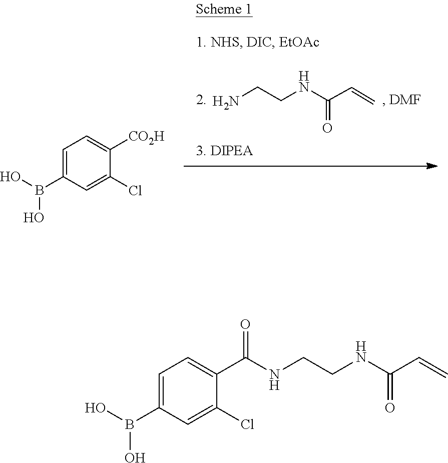

3-3-chloro-4-carboxyphenylboronic acid (0.51 g, 2.52 mmol) and N-Hydroxy-succinimide, NHS (0.32 g, 2.78 mmol) are charged into a 40 mL vial equipped with magnetic stirrer and rubber septum. Ethyl Acetate (15 mL) is added through a septum via syringe. The content of the vial is cooled down to 0° C. in ice-water bath. Diisopropyl-carbodiimide, DIC (0.35 g, 2.77 mmol) dissolved in 2 mL of ethyl acetate is added dropwise to the cooled solution of 3-chloro-4-carboxyphenylboronic acid and NHS in ethyl acetate. Ice-water bath is removed after 30 min and reaction mixture is allowed to stir overnight at room temperature. Urea precipitate which is formed is filtered off and ethyl acetate is evaporated. The active ester is redissolved in DMF (10 mL) and 2-(aminoethyl)-acrylamide×HCl (0.45 g, 2.99 mmol) is added and stirred for 10 min until all solid dissolves. The content of the reaction flask is cooled down to 0° C. in ice-water bath. N,N-Diisopropyl-ethylamine, DIPEA (0.93 g, 7.20 mmol) combined with 2 mL of DMF is added dropwise to the vial containing NHS-ester and 2-(aminoethyl)acrylamide×HCl. Ice-water bath is removed after 10 min and reaction mixture is allowed to stir for 5 h at room temperature. TLC shows consumption of NHS-ester. DMF is removed on high vacuum pump overnight and crude reaction mixture is purified by silica gel column chromatography (1.5 in×12 in; CHCl₃→CHCl₃:MeOH 100→90:10). Solvent is removed by rotary evaporation and final product is purified by crystallization from MeOH/water mixture. Crystallization over 3 days at room temperature produced white solid which is collected by filtration on Büchner funnel. It is transferred to vial and dried by oil pump vacuum overnight to give 0.372 g (1.25 mmol, 50% yield) of the final product as a white solid.

¹H-NMR (600 MHZ, DMSO-d₆+D₂O): δ=8.45 (s, 1H), 8.18 (s, 1H), 7.81 (s, 1H), 7.73 (d, 2H, J=8.0 Hz), 7.41 (d, 2H, J=8.0 Hz), 6.20 (dd, 1H, J=17.0, 10.0 Hz), 6.09 (d, 1H, J=17.0 Hz), 5.60 (d, 1H, J=10.0 Hz), 3.48 (s, 4H).

### Example 2

In current Systane products the mechanism of action relies on the formation of hydroxypropyl-guar (HP-Guar) gel with borate to prolong the retention of demulcents, such as polyethylene glycol and propylene glycol, on the eye. This helps to protect the ocular surface, thereby reducing the symptoms of the dry eye disease. The challenge is that the current retention time of demulcents on eye is not long enough as patients would like to experience.

It is believed that a water-soluble hydrophilic copolymer of the invention can be crosslinked with mucin presented on membrane bound mucins, primarily through reversible cyclo-boronate-ester linkages each formed between one substituted (carbamoylphenyl) boronic acid of the hydrophilic copolymer and one of the cis-diol moieties of the mucin and secondarily through non-covalent interactions such as hydrogen bonds formed between the hydrophilic copolymer and the mucin. As such, a water-soluble hydrophilic copolymer of the invention can act as active ingredients/lubricants and will effectively bind to membrane bound mucins.

It is also believed that when one solution of a water-soluble hydrophilic polymer of the invention is mixed with one solution of a mucin on the eye are mixed, the interactions between a water-soluble hydrophilic polymer of the invention and mucin presented on membrane bound mucins can yield an enhanced viscosity relative to the sum of the viscosities of the two solutions. Such an enhanced viscosity can be a quantitative measure of the polymer-mucin interactions and is designated in this application as a polymer-mucin-interaction synergy.

The mucin selected for the evaluation of interactions between a water-soluble hydrophilic copolymer and mucin is porcine mucin due to its similarity to human eye mucin. The mucin from porcine stomach, type III, is ordered from Sigma-Aldrich, catalogue number M1778. The received mucin is purified by ultrafiltration before it is used for this polymer-mucin interaction evaluation.

In this application, the polymer-mucin-interaction synergy (designated as PMIS) of a water-soluble hydrophilic copolymer of the invention is calculated according to Eq. (1)

$$PMIS \% = \frac{{µ}_{mix} - [ {µ}_{hc} + {µ}_{mucin} ]}{[ {µ}_{hc} + {µ}_{mucin} ]} \qquad ( 1 )$$

in which μ_{hc} is the viscosity of a first copolymer solution comprising 1.0% by weight of the water-soluble hydrophilic copolymer in phosphate-buffered saline, μ_{mucin} is the viscosity of a first mucin solution comprising 0.6% by weight of mucin type III from porcine stomach in phosphate-buffered saline, μₘᵢₓ is the viscosity of a copolymer-mucin solution obtained by mixing a second copolymer solution with a second mucin solution in equal volumes, wherein the second copolymer solution contains 2.0% by weight of the water-soluble hydrophilic copolymer in phosphate-buffered saline and the second mucin solution contains 1.2% by weight of mucin type III from porcine stomach in phosphate-buffered saline.

### Assays

MUC2 stock solution (2wt %) is prepared by dissolving freeze-dried MUC2 in DPBS using speed-mixer (1500-2000 RPM/10 min/2-3 times). A stock solution (2wt %) of a water-soluble hydrophilic copolymer of the invention is prepared by reconstituting and homogenizing the water-soluble hydrophilic copolymer in in DPBS using speed-mixer (1500-2000 RPM/10 min/2-3 times).

1.2wt % MUC2 solution is prepared from 2wt % MUC2 stock solution by dilution with DPBS.

Equal volume (1 mL) of 1.2wt % MUC2 solution and DPBS are mixed in 20 mL scintillation vial to form a 0.6wt % MUC2 solution. The prepared 0.6wt % MUC2 solution is tested for viscosity according to the procedures described below.

Equal volume (1 mL) of 2wt % hydrophilic copolymer solution and DPBS are mixed in 20 mL scintillation vial to form a 1wt % copolymer solution. The prepared 1wt % copolymer solution is tested for viscosity according to the procedures described below.

2 mL of 2.0% copolymer solution is added to a 20 mL scintillation vial with a stir bar. Vial is placed on stir plate set at 500-600 RPM. 2 mL of 1.2% MUC2 solution is added dropwise over the span of 10-20 seconds. Check the mixed solution which does show free flow as a homogenous viscous solution without cluster or coagulated gel. Solution is mixed at 500-600 RPM for 30 min, then mixing speed is reduced to 150-200 RPM for 20-30 min to dissipate bubbles.

Viscosity measurements are carried out in a DVNext Wells-Brookfield Cone/Plate Rheometer as follows. About 0.5 mL of a sample is placed into the sample cup. All viscosities are measured with size 40 spindle at room temperature.

### Example 3

### Copolymer Synthesis

A stock solution of CPBA (4 wt %) is prepared by dissolving a desired amount of CPBA in DMSO; stock solution of Vazo-56 (1wt %), MBA (1wt %), and MPC (17wt %) are prepared by dissolving a desired amount of Vazo-56, MBA or MPC in D.I. water.

Combine the stock solutions with the quantities which meet the target amounts as shown in Table 1, directly in a jacketed reactor equipped with an overhead stirrer, a condenser, thermocouple, and a nitrogen gas dispersion fritted tube. Then, fill the remaining part with water to make a final 5% solid solution ([Vazo-56]=2,2′-azobis(2-methylpropionamidine) dihydrochloride).

TABLE 1

| | | Composition, wt % (% by mole) | | | | | |
|---|---|---|---|---|---|---|---|
| | | 3-1 | 3-2 | 3-3 | 3-4 | 3-5 | 3-6 |
| | | P(DMA- | P(DMA- | P(DMA-AA- | P(HEAm- | P(DMA-MPC- | P(HEAm-MPC- |
| Monomer | MW | APBA) | CPBA) | CPBA) | CPBA) | CPBA) | CPBA) |
| DMA | 99.1 | 89.9 (94.5) | 86.2 (94.5) | 83.9 (84.0) | — | 49.5 (74.5) | — |
| HEAm | 113.1 | — | — | — | 87.0 (94.5) | — | 53.2 (74.5) |
| MPC | 295.3 | — | — | — | — | 39.6 (20.0) | 36.7 (20.0) |
| APBA | 191.0 | 10.1 (5.5) | — | — | — | — | — |
| CPBA | 296.5 | — | 14.8 (5.5) | 5.9 (2.0) | 13.0 (5.5) | 10.9 (5.5) | 10.1 (5.5) |
| AA | | | | 10.2 (14.0) | | | |
| MBA | 154.2 | — | — | — | 0.06 (0.06) | 0.12 (0.12) | 0.12 (0.12) |

First value represents wt %, values in parentheses represent mole %.

Purge a polymerizable composition with nitrogen to remove oxygen. Left the frit of the gas dispersion tube above the polymerizable composition and the maintain a nitrogen flow around 50 mL/min throughout the polymerization. Ramp the polymerizable composition's temperature from 16° C. to 56° C. over 2 hours. After 10 hours, lower the temperature from 56° C. to 20° C. over 2 hours. Purify the crude polymer solution by ultrafiltration using a 1 M Dalton cut-off membrane to remove the residual monomers, low MW polymers, and any residual solvent. Freeze-dry purified polymer for further characterization and testing.

The purified polymers are characterized. The results are reported in Table 2.

TABLE 2

| | Copolymer Characterization | | | | | |
|---|---|---|---|---|---|---|
| | 3-1 | 3-2 | 3-3 | 3-4 | 3-5 | 3-6 |
| | P(DMA- | P(DMA- | P(DMA-AA- | P(HEAm- | P(DMA- | P(HEAm-MPC- |
| | APBA) | CPBA) | CPBA) | CPBA) | MPC-CPBA) | CPBA) |
| Yield _{UF w/ 1M} | 98% | 94% | 87% | 96% | 74% | 88% |
| Viscosity (cP) _{0.5% in PBS} | 27 | 136 | 62 | 51 | 15 | 23 |
| FGC _{meq/g} * | 0.51 (_{0.52}) | 0.55 (_{0.50}) | TBD | 0.45 (_{0.44}) | 0.42 (_{0.37}) | 0.39 (_{0.34}) |
| M_{w} × 10⁶ | TBD | TBD | TBD | TBD | TBD | TBD |
| (g/mol) _{D} | | | | | | |

* the content of phenylboronic acid; and the values in parenthesis are the theoretical values based on the composition of the polymerizable composition.

### Example 4

Water-soluble hydrophilic copolymers are prepared according to the procedures described in Example 3 and characterized. The composition of the polymerizable compositions for preparing copolymers and characterization results are reported in Table 3.

TABLE 3

| | 3-5 | 4-1 | 4-2 | 4-3 |
|---|---|---|---|---|
| Monomer | P(DMA-MPC-CPBA) | P(DMA-MPC-CPBA) | P(DMA-MPC-CPBA) | P(DMA-MPC-CPBA) |
| DMA | 49.5% (74.5%) | 54.3% (78.0%) | 56.6% (79.5%) | 57.2% (79.95%) |
| MPC | 39.6% (20%) | 41.5% (20.0%) | 42.4% (20.0%) | 42.7% (20%) |
| CPBA | 10.9% (5.5%) | 4.20% (4.16%) | 1.00% (0.5%) | 0.10% (0.05%) |
| MBA | 0.12% (0.12%) | — | — | — |

Basic Copolymer Properties

| | | | | |
|---|---|---|---|---|
| Yield_{UF w/1M} | 74% | 90% | 85% | 92% |
| Viscosity (cP)_{0.5% in PBS} | 15 | 22 | 39 | 35 |
| FGC_{meq/g} | 0.42_{0.36} | 0.21_{0.14} | 0.14_{0.04} | n/a |
| M_{w} * 10⁶ | TBD | TBD | TBD | TBD |
| (g/mol)_{D} | | | | |

First value represents wt %, values in parentheses represent mole %.

### Example 5

Water-soluble hydrophilic copolymers are prepared according to the procedures described in Example 3 and characterized. The composition of the polymerizable compositions for preparing copolymers and characterization results are reported in Table 4.

TABLE 4

| | 3-6 | 5-1 | 5-2 | 5-3 |
|---|---|---|---|---|
| Monomer | P(HEAm-MPC-CPBA) | P(HEAm-MPC-CPBA) | P(HEAm-MPC-CPBA) | P(HEAm-MPC-CPBA) |
| HEAm | 53.2% (74.5%) | 58.0% (78.0%) | 60.2% (79.5%) | 60.9% (79.95%) |
| MPC | 36.7% (20%) | 38.2% (20.0%) | 38.8% (20.0%) | 39.0% (20.0%) |
| CPBA | 10.1% (5.5%) | 3.80% (2.0%) | 1.00% (0.5%) | 0.10% (0.05%) |
| MBA | 0.12% (0.12%) | 0.06% (0.06%) | 0.12% (0.12%) | 0.12% (0.12%) |

Basic Copolymer Properties

| | | | | |
|---|---|---|---|---|
| Yield_{UF w/1M} | 88% | 91% | 91% | 88% |
| Viscosity (cP)_{0.5% in PBS} | 23 | 26 | 36 | 62 |
| FGC_{meq/g} | 0.39_{0.34} | 0.26_{0.13} | 0.14_{0.03} | 0.11_{0.003} |
| M_{w} * 10⁶ | TBD | TBD | TBD | TBD |
| (g/mol)_{D} | | | | |

First value represents wt %, values in parentheses represent mole %.

### Example 6

Water-soluble hydrophilic copolymers are prepared according to the procedures described in Example 3 and characterized. The composition of the polymerizable compositions for preparing copolymers and characterization results are reported in Table 5.

TABLE 5

| | 6-1 | 6-2 | 6-3 | 6-4 |
|---|---|---|---|---|
| Monomer | P(HEAm-MPC-CPBA) | P(HEAm-MPC-CPBA) | P(HEAm-MPC-CPBA) | P(HEAm-MPC-CPBA) |
| HEAm | 74.1% (88.0%) | 58.0% (78.0%) | 58.0% (78.0%) | 45.3% (68.0%) |
| MPC | 21.5% (10.0%) | 38.2% (20.0%) | 38.2% (20.0%) | 51.2% (30.0%) |
| CPBA | 4.4% (2.0%) | 3.8% (2.0%) | 3.8% (2.0%) | 3.5% (2.0%) |
| MBA | 0.06% (0.06%) | 0.06% (0.06%) | 0.06% (0.06%) | 0.06% (0.06%) |

Basic Copolymer Properties

| | | | | |
|---|---|---|---|---|
| Yield_{UF w/1M} | 90% | 93% | 93% | 93% |
| Viscosity (cP)_{0.5% in PBS} | 47 | 24 | 20 | 26 |
| FGC_{meq/g} | 0.22_{0.14} | 0.19_{0.15} | 0.22_{0.15} | 0.20_{0.12} |
| M_{w} × 10⁶ | TBD | TBD | TBD | TBD |
| (g/mol)_{D} | | | | |

First value represents wt %, values in parentheses represent mole %.

### Example 7

The polymer-mucin-interaction synergies (PMISs) of the water-soluble hydrophilic copolymers prepared in Example 3 are determined according to the procedures described in Example 2. The results are reported in Tables 6 and 7.

TABLE 6

| | Copolymer (mole %) | |
|---|---|---|
| | 3-1 | 3-2 |
| | P(DMA-APBA) | P(DMA-CPBA) |
| DMA | 94.5 | 94.5 |
| APBA | 5.5 | — |
| CPBA | — | 5.5 |
| Theoretical Mixed Viscosity | 7.80 | 8.75 |
| (cP) | | |
| Measured Viscosity (cP) | 9.14 | 46.02 |
| PMIS (%) | 17% | 426% |

TABLE 7

| | 3-2 | 3-4 | 3-5 | 3-6 |
|---|---|---|---|---|
| | P(DMA-CPBA) | P(HEAm-CPBA) | P(DMA-MPC-CPBA) | P(HEAm-MPC-CPBA) |
| DMA (mole %) | 94.5 | — | 74.5 | — |
| HEAm (mol %) | — | 94.5 | — | 74.5 |
| MPC (mole %) | — | — | 20 | 20 |
| CPBA (mole %) | 5.5 | 5.5 | 5.5 | 5.5 |
| Theoretical Mixed Viscosity (cP) | 8.75 | 7.66 | 9.85 | 7.57 |
| Measured Viscosity (cP) | 46.02 | 140.80 | 33.57 | 42.97 |
| PMIS (%) | 426% | 1739% | 241% | 468% |

Table 6 shows the comparison of the strength of polymer-mucin interactions of a copolymer of the invention (having repeating monomeric units of CPBA with a pKa of about 7.2) to that of a control copolymer (having repeating monomeric units of APBA with a pKa of about 8.1). A water-soluble hydrophilic copolymer of the invention has stronger mucin interactions than the control copolymer.

Table 7 shows that replacing DMA (N_{H-bond doner}−N_{H-bond acceptor}=0−1=−1) in the polymer backbone with HEAm (N_{H-bond doner}−N_{H-bond acceptor}=2−2=0) significantly enhances polymer-mucin interactions. Viscosity increases from 46 cP (DMA) to 141 cP (HEAm), which translates an PMIS and 426% and 1739% respectively. This result may be hydrogen bonding between HEAm-containing copolymer and mucin due to the presence of H-bond donors in HEAm. On the other hand, addition of MPC (20 mol %) to either copolymer reduces PMIS, i.e., polymer-mucin interactions (Table 7). 2-Methacryloyloxyethyl phosphorylcholine (MPC) may attenuate the mucin interaction, but HEAm seems help to regain the interaction strength.

### Example 8

The polymer-mucin-interaction synergies (PMISs) of the water-soluble hydrophilic copolymers prepared in Example 4 are determined according to the procedures described in Example 2. The results are reported in Table 8.

TABLE 8

| | 3-5 | 4-1 | 4-2 | 4-3 |
|---|---|---|---|---|
| DMA (mole %) | 74.5 | 78 | 79.5 | 79.95 |
| MPC (mole %) | 20 | 20 | 20 | 20 |
| CPBA (mole %) | 5.5 | 2.0 | 0.50 | 0.05 |
| Theoretical Mixed Viscosity (cP) | 10.50 | 8.17 | 9.56 | 7.81 |
| Measured Viscosity (cP) | 34.66 | 13.24 | 12.33 | 11.27 |
| PMIS (%) | 230% | 62% | 29% | 35% |

Table 8 shows the effects of the content of the repeating monomeric units of CPBA on PMIS (polymer-mucin interaction strength) of poly(DMA-MPC-CPBA). The higher the CPBA content, the higher the PMIS (i.e., the stronger the polymer-mucin interactions).

### Example 9

The polymer-mucin-interaction synergies (PMISs) of the water-soluble hydrophilic copolymers prepared in Example 5 are determined according to the procedures described in Example 2. The results are reported in Table 9.

TABLE 9

| | 3-6 | 5-1 | 5-2 | 5-3 |
|---|---|---|---|---|
| HEAm (mole %) | 74.5 | 78 | 79.5 | 79.95 |
| MPC (mole %) | 20 | 20 | 20 | 20 |
| CPBA (mole %) | 5.5 | 2.0 | 0.50 | 0.05 |
| MBA (mole %) | 0.12 | 0.06 | 0.12 | 0.12 |
| Theoretical Mixed Viscosity (cP) | 8.63 | 8.43 | 10.55 | 13.75 |
| Measured Viscosity (cP) | 47.42 | 14.59 | 15.43 | 18.35 |
| PMIS (%) | 450% | 73% | 46% | 34% |

Table 9 shows the effects of the content of the repeating monomeric units of CPBA on PMIS (polymer-mucin interaction strength) of poly(HEAm-MPC-CPBA). The higher the CPBA content, the higher the PMIS (i.e., the stronger the polymer-mucin interactions).

### Example 10

The polymer-mucin-interaction synergies (PMISs) of the water-soluble hydrophilic copolymers prepared in Example 6 are determined according to the procedures described in Example 2. The results are reported in Table 10.

TABLE 10

| | 6-1 | 6-2 | 6-3 | 6-4 |
|---|---|---|---|---|
| HEAm (mole %) | 88 | 78 | 78 | 68 |
| MPC (mole %) | 10 | 20 | 20 | 30 |
| CPBA (mole %) | 2.0 | 2.0 | 2.0 | 2.0 |
| MBA (mole %) | 0.06 | 0.06 | 0.06 | 0.06 |
| Theoretical Mixed Viscosity (cP) | 12 | 8 | 9 | 8 |
| Measured Viscosity (cP) | 22.90 | 13.46 | 14.96 | 11.41 |
| PMIS (%) | 94% | 61% | 71% | 51% |

Table 10 shows the effects of the content of the repeating monomeric units of MPC on PMIS (polymer-mucin interaction strength) of poly(HEAm-MPC-CPBA). The higher the MPC content, the lower the PMIS (i.e., the stronger the polymer-mucin interactions).

All the publications, patents, and patent application publications, which have been cited herein above in this application, are hereby incorporated by reference in their entireties.

### Embodiments

The present disclosure is further directed to the following embodiments which may be combined with any embodiments described herein.

Clause 1. A water-soluble hydrophilic copolymer, comprising: from about 0.01% to about 7.0% by mole of repeating monomeric units of at least one arylborono-containing vinylic monomer having an arylborono group and a pKa of from about 6.4 to about 7.8; from about 60.0% to 99.9% by mole of repeating monomeric units of at least one hydrophilic vinylic monomer which is free of phosphorylcholine group and has N_{H-bond donner}−N_{H-bond acceptor}≥2−1 in which N_{H-bond donner} and N_{H-bond acceptor} are the H-bond donner and H-bond acceptor counts respectively of the hydrophilic vinylic monomer; from 0% to about 35% by mole of repeating monomeric units of at least one phosphorylcholine-containing vinylic monomer; and from 0% to about 0.20% by mole of repeating units of at least one hydrophilic vinylic crosslinker, wherein the water-soluble hydrophilic copolymer has a polymer-mucin-interaction synergy of at least 50%, wherein the polymer-mucin-interaction synergy (designated as PMIS) is calculated according to Eq. (1)

$$PMIS \% = \frac{{µ}_{mix} - [ {µ}_{hc} + {µ}_{mucin} ]}{[ {µ}_{hc} + {µ}_{mucin} ]}$$

in which μ_{hc} is the viscosity of a first copolymer solution comprising 1.0% by weight of the water-soluble hydrophilic copolymer in phosphate-buffered saline, μ_{mucin} is the viscosity of a first mucin solution comprising 0.6% by weight of mucin type III from porcine stomach in phosphate-buffered saline, μₘᵢₓ is the viscosity of a copolymer-mucin solution obtained by mixing a second copolymer solution with a second mucin solution in equal volumes, wherein the second copolymer solution contains 2.0% by weight of the water-soluble hydrophilic copolymer in phosphate-buffered saline and the second mucin solution contains 1.2% by weight of mucin type III from porcine stomach in phosphate-buffered saline.

Clause 2. The water-soluble hydrophilic copolymer of Clause 1, wherein the water-soluble hydrophilic copolymer comprises from about 0.1% to about 6.0% by mole of the repeating monomeric units of said at least one arylborono-containing vinylic monomer. Clause 3. The water-soluble hydrophilic copolymer of Clause 1, wherein the water-soluble hydrophilic copolymer comprises from about 0.5% to about 5.0% by mole of the repeating monomeric units of said at least one arylborono-containing vinylic monomer. Clause 4. The water-soluble hydrophilic copolymer of Clause 1, wherein the water-soluble hydrophilic copolymer comprises from about 1.0% to about 4.0% by mole of the repeating monomeric units of said at least one arylborono-containing vinylic monomer.

Clause 5. The water-soluble hydrophilic copolymer of any one of Clauses 1 to 4, wherein said at least one arylborono-containing vinylic monomer has a pKa of from about 6.8 to about 7.6.

Clause 6. The water-soluble hydrophilic copolymer of any one of Clauses 1 to 4, wherein said at least one arylborono-containing vinylic monomer has a pKa of from about 6.9 to about 7.5.

Clause 7. The water-soluble hydrophilic copolymer of any one of Clauses 1 to 6, wherein the water-soluble hydrophilic copolymer comprises from about 60% to about 94.9% by mole of the repeating monomeric units of said at least one hydrophilic vinylic monomer. Clause 8. The water-soluble hydrophilic copolymer of any one of Clauses 1 to 6, wherein the water-soluble hydrophilic copolymer comprises from about 65% to about 94% by mole of the repeating monomeric units of said at least one hydrophilic vinylic monomer.

Clause 9. The water-soluble hydrophilic copolymer of any one of Clauses 1 to 6, wherein the water-soluble hydrophilic copolymer comprises from about 70% to about 93% by mole of the repeating monomeric units of said at least one hydrophilic vinylic monomer. Clause 10. The water-soluble hydrophilic copolymer of any one of Clauses 1 to 9, wherein the water-soluble hydrophilic copolymer comprises from about 5% to about 30% by mole of the repeating monomeric units of said at least one phosphorylcholine-containing vinylic monomer.

Clause 11. The water-soluble hydrophilic copolymer of any one of Clauses 1 to 9, wherein the water-soluble hydrophilic copolymer comprises from about 10% to about 25% by mole of the repeating monomeric units of said at least one phosphorylcholine-containing vinylic monomer.

Clause 12. The water-soluble hydrophilic copolymer of any one of Clauses 1 to 11, wherein the water-soluble hydrophilic copolymer comprises from 0% to about 0.15% by mole of the repeating units of said at least one hydrophilic vinylic crosslinker.

Clause 13. The water-soluble hydrophilic copolymer of any one of Clauses 1 to 11, wherein the water-soluble hydrophilic copolymer comprises from 0% to about 0.1% by mole of the repeating units of said at least one hydrophilic vinylic crosslinker.

Clause 14. The water-soluble hydrophilic copolymer of any one of Clauses 1 to 13, wherein said at least one arylborono-containing vinylic monomer comprises 4-(1,6-Dioxo-2,5-diaza-7-oxamyl)phenylboronic acid, 2-dimethylamino-methyl-5-vinylphenylboronic acid, 4-(N-allylsulfamoyl)phenylboronic acid, 4-(3-Butenylsulfonyl)phenylboronic acid, 4-acrylamido-3-chlorophenylboronic acid, 3-(meth)acrylamido-5-nitrophenylboronic acid, 4-(meth)acrylamido-5-nitrophenylboronic acid, 4-(meth)acrylamido-3-nitrophenylboronic acid, 3-(meth)acrylamido-6-hydroxymethylphenylboronic acid, 3-(meth)acrylamido-6-dimethylaminomethylphenylboronic acid, 4-(meth)acrylamido-6-hydroxymethylphenylboronic acid, 4-(meth)acrylamido-6-dimethylaminomethylphenylboronic acid, or a combination thereof.

Clause 15. The water-soluble hydrophilic copolymer of any one of Clauses 1 to 13, wherein said at least one arylborono-containing vinylic monomer comprises an arylborono-containing acrylamido monomer having a (carbamoylphenyl)boronic acid linked to a (meth)acrylamido group.

Clause 16. The water-soluble hydrophilic copolymer of Clause 15, wherein the arylborono-containing acrylamido monomer is represented by formula (I)

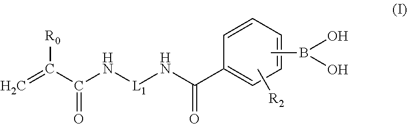

in which R₀ is H or CH₃, R₂ is NO₂, F, Cl, Br, CF₃, CCl₃, or CH₂N(CH₃)₂, and L₁ is a C₂-C₆ alkylene divalent radical or a divalent radical —R₃—X₁—R₄— in which X₁ is an amide linkage of —C(O)NH—, R₃ and R₄ independent of each other are a C₂-C₆ alkylene divalent radical which is optionally substituted with one or more hydroxyl groups, wherein the acrylamido monomer has a pKa of from about 6.4 to about 7.6.

Clause 17. The water-soluble hydrophilic copolymer of Clause 16, wherein in formula (I) the boronic acid group of *—B(OH)₂ is at para position.

Clause 18. The water-soluble hydrophilic copolymer of Clauses 16 or 17, wherein in formula (I) L₁ is a divalent radical —R₃—X₁—R₄— in which X₁ is an amide linkage of —C(O)NH—, R₃ and R₄ independent of each other are a C₂-C₆ alkylene divalent radical which is optionally substituted with one or more hydroxyl groups.

Clause 19. The water-soluble hydrophilic copolymer of Clauses 16 or 17, wherein in formula (I) L₁ is a C₂-C₆ alkylene divalent radical.

Clause 20. The water-soluble hydrophilic copolymer of any one of Clauses 16 to 19, wherein in formula (I) R₂ is Cl.

Clause 21. The water-soluble hydrophilic copolymer of any one of Clauses 16 to 19, wherein in formula (I) R₂ is NO₂.

Clause 22. The water-soluble hydrophilic copolymer of Clause 16, wherein the arylborono-containing acrylamido monomer is

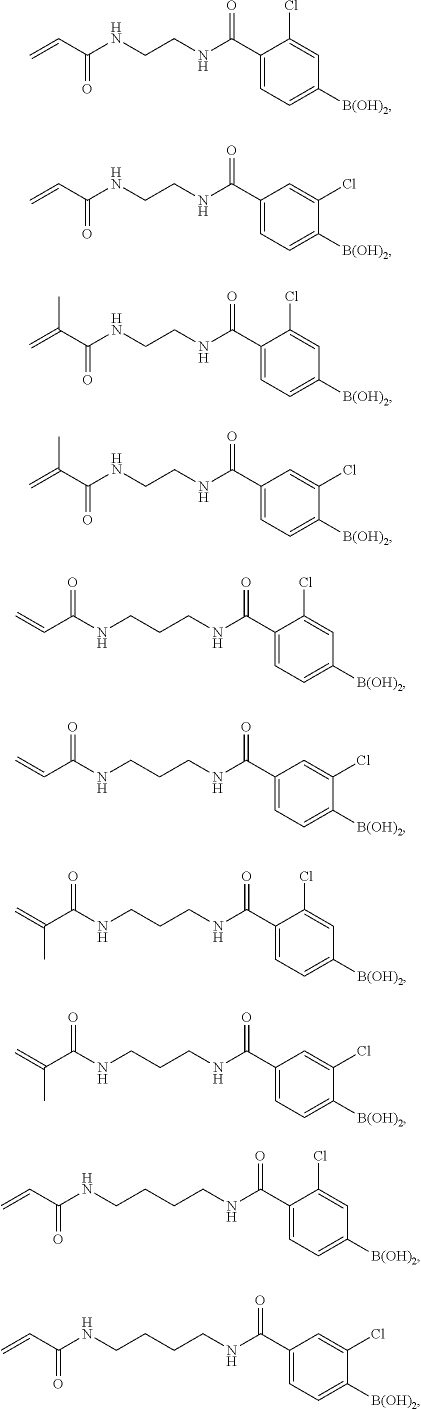

or combinations thereof.

Clause 23. The water-soluble hydrophilic copolymer of Clause 16, wherein the arylborono-containing acrylamido monomer is

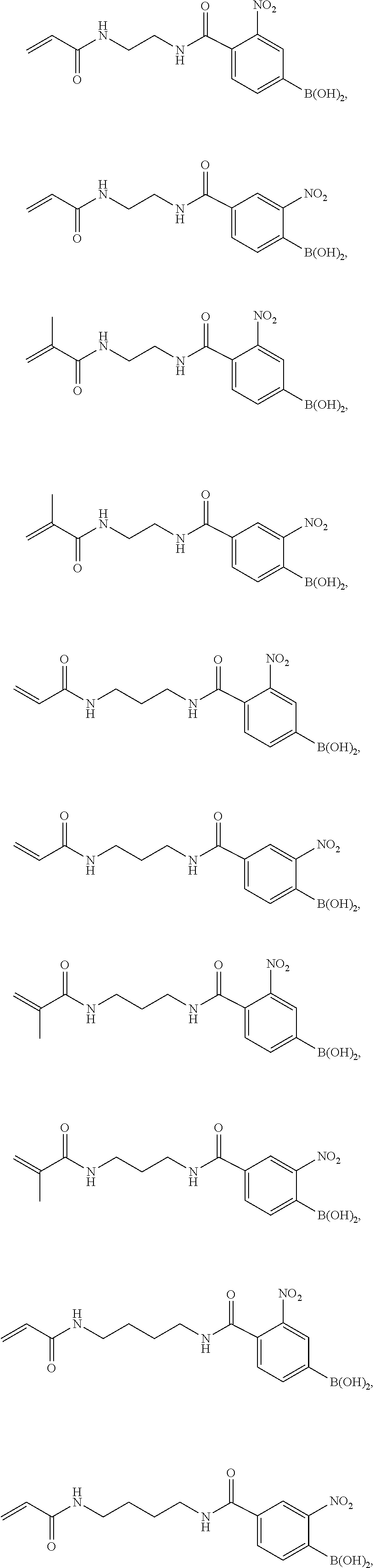

or combinations thereof.

Clause 24. The water-soluble hydrophilic copolymer of any one of Clauses 1 to 23, wherein said at least one hydrophilic vinylic monomer comprises a hydrophilic acrylamido monomer, an N-vinyl amide monomer, a methylene-containing pyrrolidone monomer, N-2-hydroxyethyl vinyl carbamate, (meth)acrylic acid, ethylacrylic acid, propylacrylic acid, or combinations thereof.

Clause 25. The water-soluble hydrophilic copolymer of any one of Clauses 1 to 24, wherein said at least one hydrophilic vinylic monomer comprises (meth)acrylamide, N,N-dimethyl (meth)acrylamide, N,N-diethyl (meth)acrylamide, N-ethyl (meth)acrylamide, N-propyl (meth)acrylamide, N-isopropyl (meth)acrylamide, N-2-hydroxylethyl (meth)acrylamide, N,N-bis(hydroxyethyl) (meth)acrylamide, N-3-hydroxypropyl (meth)acrylamide, N-2-hydroxypropyl (meth)acrylamide, N-2-aminoethyl (meth)acrylamide, N-2-methylaminoethyl (meth)acrylamide, N-2-ethylaminoethyl (meth)acrylamide, N-2-dimethylaminoethyl (meth)acrylamide, N-3-aminopropyl (meth)acrylamide, N-3-methylamino-propyl (meth)acrylamide, 2-(meth)acrylamidoglycolic acid, 2-(meth)acrylamidopropionic acid, 3-(meth)acrylamidopropionic acid, 4-(meth)acrylamidobutanoic acid, 5-(meth)acrylamido-pentanoic acid, or combinations thereof.

Clause 26. The water-soluble hydrophilic copolymer of any one of Clauses 1 to 25, wherein said at least one hydrophilic vinylic monomer comprises meth)acrylamide, N,N-dimethyl (meth)acrylamide, N-ethyl (meth)acrylamide, N-propyl (meth)acrylamide, N-isopropyl (meth)acrylamide, N-2-hydroxylethyl (meth)acrylamide, N,N-bis(hydroxyethyl) (meth)acrylamide, N-3-hydroxypropyl (meth)acrylamide, N-2-hydroxypropyl (meth)acrylamide, or combinations thereof.

Clause 27. The water-soluble hydrophilic copolymer of any one of Clauses 1 to 26, wherein said at least one hydrophilic vinylic monomer comprises (meth)acrylic acid, ethylacrylic acid, propylacrylic acid, or combinations thereof.

Clause 28. The water-soluble hydrophilic copolymer of any one of Clauses 1 to 27, wherein said at least one hydrophilic vinylic monomer comprises N-vinylpyrrolidone, N-vinyl-3-methyl-2-pyrrolidone, N-vinyl-4-methyl-2-pyrrolidone, N-vinyl-5-methyl-2-pyrrolidone, N-vinyl-6-methyl-2-pyrrolidone, N-vinyl-3-ethyl-2-pyrrolidone, N-vinyl-4,5-dimethyl-2-pyrrolidone, N-vinyl-5,5-dimethyl-2-pyrrolidone, N-vinyl-3,3,5-trimethyl-2-pyrrolidone, N-vinyl piperidone, N-vinyl-3-methyl-2-piperidone, N-vinyl-4-methyl-2-piperidone, N-vinyl-5-methyl-2-piperidone, N-vinyl-6-methyl-2-piperidone, N-vinyl-6-ethyl-2-piperidone, N-vinyl-3,5-dimethyl-2-piperidone, N-vinyl-4,4-dimethyl-2-piperidone, N-vinyl caprolactam, N-vinyl-3-methyl-2-caprolactam, N-vinyl-4-methyl-2-caprolactam, N-vinyl-7-methyl-2-caprolactam, N-vinyl-7-ethyl-2-caprolactam, N-vinyl-3,5-dimethyl-2-caprolactam, N-vinyl-4,6-dimethyl-2-caprolactam, N-vinyl-3,5,7-trimethyl-2-caprolactam, N-vinyl-N-methyl acetamide, N-vinyl formamide, N-vinyl acetamide, N-vinyl isopropylamide, N-vinyl-N-ethyl acetamide, N-vinyl-N-ethyl formamide, or combinations thereof (preferably N-vinylpyrrolidone, N-vinyl-N-methyl acetamide, or combinations thereof).

Clause 29. The water-soluble hydrophilic copolymer of any one of Clauses 1 to 28, wherein said at least one hydrophilic vinylic monomer comprises 1-methyl-3-methylene-2-pyrrolidone, 1-ethyl-3-methylene-2-pyrrolidone, 1-methyl-5-methylene-2-pyrrolidone, 1-ethyl-5-methylene-2-pyrrolidone, 5-methyl-3-methylene-2-pyrrolidone, 5-ethyl-3-methylene-2-pyrrolidone, 1-n-propyl-3-methylene-2-pyrrolidone, 1-n-propyl-5-methylene-2-pyrrolidone, 1-isopropyl-3-methylene-2-pyrrolidone, 1-isopropyl-5-methylene-2-pyrrolidone, 1-n-butyl-3-methylene-2-pyrrolidone, 1-tert-butyl-3-methylene-2-pyrrolidone, allyl alcohol, or combinations thereof.

Clause 30. The water-soluble hydrophilic copolymer of any one of Clauses 1 to 29, wherein said at least one phosphorylcholine-containing vinylic monomer comprises (meth)acryloyloxyethyl phosphorylcholine, (meth)acryloyloxypropyl phosphorylcholine, 4-((meth)acryloyloxy)butyl-2′-(trimethylammonio)ethylphosphate, 2-[(meth)acryloylamino]-ethyl-2′-(trimethylammonio)-ethylphosphate, 3-[(meth)acryloylamino]propyl-2′-(trimethyl-ammonio)ethylphosphate, 4-[(meth)acryloylamino]butyl-2′-(trimethylammonio)ethyl-phosphate, 5-((meth)acryloyloxy)pentyl-2′-(trimethylammonio)ethyl phosphate, 6-((meth)acryloyloxy)hexyl-2′-(trimethylammonio)-ethylphosphate, 2-((meth)acryloyloxy)ethyl-2′-(triethylammonio)ethylphosphate, 2-((meth)acryloyloxy)ethyl-2′-(tripropylammonio)ethyl-phosphate, 2-((meth)acryloyloxy)ethyl-2′-(tributylammonio)ethyl phosphate, 2-((meth)acryloyloxy)propyl-2′-(trimethylammonio)-ethylphosphate, 2-((meth)acryloyloxy)-butyl-2′-(trimethylammonio)ethylphosphate, 2-((meth)acryloyloxy)pentyl-2′-(trimethylammonio)ethylphosphate, 2-((meth)acryloyloxy)hexyl-2′-(trimethylammonio) ethyl phosphate, 2-(vinyloxy)ethyl-2′-(trimethylammonio)ethylphosphate, 2-(allyloxy)ethyl-2′-(trimethylammonio)ethylphosphate, 2-(vinyloxycarbonyl)ethyl-2′-(trimethylammonio)ethyl phosphate, 2-(allyloxycarbonyl)ethyl-2′-(trimethylammonio)-ethylphosphate, 2-(vinylcarbonylamino)ethyl-2′-(trimethylammonio)ethylphosphate, 2-(allyloxycarbonylamino)-ethyl-2′-(trimethylammonio)ethyl phosphate, 2-(butenoyloxy)ethyl-2′-(trimethylammonio)-ethylphosphate, or combinations thereof. Clause 31. The water-soluble hydrophilic copolymer of any one of Clauses 1 to 29, wherein said at least one hydrophilic vinylic crosslinker comprises ethyleneglycol di-(meth)acrylate, diethyleneglycol di-(meth)acrylate, triethyleneglycol di-(meth)acrylate, tetraethyleneglycol di-(meth)acrylate, polyethylene glycol di-(meth)acrylate having a number averaged molecular weight of from 200 to 1,000 daltons, glycerol di-(meth)acrylate, glycerol 1,3-diglycerolate di-(meth)acrylate, ethylenebis[oxy(2-hydroxypropane-1,3-diyl) ]di-(meth)acrylate, bis[2-(meth)acryloxyethyl]phosphate, diacrylamide, dimethacrylamide, N,N-di(meth)acryloyl-N-methylamine, N,N-di(meth)acryloyl-N-ethylamine, N,N′-methylene bis(meth)acrylamide, N,N′-ethylene bis(meth)acrylamide, N,N′-dihydroxyethylene bis(meth)acrylamide, N,N′-propylene bis(meth)acrylamide, N,N′-2-hydroxypropylene bis(meth)acrylamide, N,N′-2,3-dihydroxybutylene bis(meth)acrylamide, 1,3-bis(meth) acrylamide-propane-2-yl dihydrogen phosphate, piperazine diacrylamide, or combinations thereof.

Clause 32. The water-soluble hydrophilic copolymer of any one of Clauses 1 to 29, wherein said at least one hydrophilic vinylic crosslinker comprises more preferably selected from the group consisting of N,N-di(meth)acryloyl-N-methylamine, N,N-di(meth)acryloyl-N-ethylamine, N,N′-methylene bis(meth)acrylamide, N,N′-ethylene bis(meth)acrylamide, N,N′-dihydroxyethylene bis(meth)acrylamide, N,N′-propylene bis(meth)acrylamide, N,N′-2-hydroxypropylene bis(meth)acrylamide, N,N′-2,3-dihydroxybutylene bis(meth)acrylamide, combinations thereof.

Clause 33. The water-soluble hydrophilic copolymer of any one of Clauses 1 to 32, wherein the water-soluble hydrophilic copolymer has a number average molecular weight of from about 10,000 Daltons to about 10,000,000 Daltons.

Clause 34. The water-soluble hydrophilic copolymer of any one of Clauses 1 to 32, wherein the water-soluble hydrophilic copolymer has a number average molecular weight of from about 25,000 Daltons to 5,000,000 Daltons.

Clause 35. The water-soluble hydrophilic copolymer of any one of Clauses 1 to 32, wherein the water-soluble hydrophilic copolymer has a number average molecular weight of from about 50,000 Daltons to about 2,000,000 Daltons.

Clause 36. An ophthalmic composition, comprising:

from about 0.1 w/v % to about 10 w/v % of a water-soluble hydrophilic copolymer of any one of Clauses 1 to 35;

one or more buffering agents in an amount sufficient to provide the ophthalmic composition with a pH of from about 6.5 to about 8.2; and optionally one or more additional excipients and/or one or more additional active ingredients.

What is claimed is:

1. A water-soluble hydrophilic copolymer, comprising: from about 0.01% to about 7.0% by mole of repeating monomeric units of at least one arylborono-containing vinylic monomer having an arylborono group and a pKa of from about 6.4 to about 7.8; from about 60.0% to 99.9% by mole of repeating monomeric units of at least one hydrophilic vinylic monomer which is free of phosphorylcholine group and has N_{H-bond doner}−N_{H-bond acceptor}≥−1 in which N_{H-bond doner} and N_{H-bond acceptor} are the H-bond doner and H-bond acceptor counts respectively of the hydrophilic vinylic monomer; from 0% to about 35% by mole of repeating monomeric units of at least one phosphorylcholine-containing vinylic monomer; and from 0% to about 0.20% by mole of repeating units of at least one hydrophilic vinylic crosslinker, wherein the water-soluble hydrophilic copolymer has a polymer-mucin-interaction synergy of at least 50%, wherein the polymer-mucin-interaction synergy (designated as PMIS) is calculated according to Eq. (1)
$$PMIS \% = \frac{{µ}_{mix} - [ {µ}_{hc} + {µ}_{mucin} ]}{[ {µ}_{hc} + {µ}_{mucin} ]}$$
in which μ_{hc} is the viscosity of a first copolymer solution comprising 1.0% by weight of the water-soluble hydrophilic copolymer in phosphate-buffered saline, μ_{mucin} is the viscosity of a first mucin solution comprising 0.6% by weight of mucin type III from porcine stomach in phosphate-buffered saline, μₘᵢₓ is the viscosity of a copolymer-mucin solution obtained by mixing a second copolymer solution with a second mucin solution in equal volumes, wherein the second copolymer solution contains 2.0% by weight of the water-soluble hydrophilic copolymer in phosphate-buffered saline and the second mucin solution contains 1.2% by weight of mucin type III from porcine stomach in phosphate-buffered saline.

2. The water-soluble hydrophilic copolymer of claim 1, wherein the water-soluble hydrophilic copolymer comprises from about 0.1% to about 6.0% by mole of the repeating monomeric units of said at least one arylborono-containing vinylic monomer.

3. The water-soluble hydrophilic copolymer of claim 1, wherein the water-soluble hydrophilic copolymer comprises from about 60% to about 94.9% by mole of the repeating monomeric units of said at least one hydrophilic vinylic monomer.

4. The water-soluble hydrophilic copolymer of claim 1, wherein the water-soluble hydrophilic copolymer comprises from about 5% to about 30% by mole of the repeating monomeric units of said at least one phosphorylcholine-containing vinylic monomer.

5. The water-soluble hydrophilic copolymer of claim 1, wherein said at least one arylborono-containing vinylic monomer comprises an arylborono-containing acrylamido monomer having a (carbamoylphenyl)boronic acid linked to a (meth)acrylamido group.

6. The water-soluble hydrophilic copolymer of claim 5, wherein the arylborono-containing acrylamido monomer is represented by formula (I) 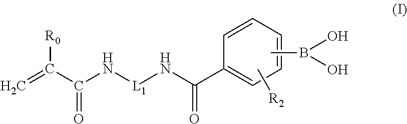
in which R₀ is H or CH₃, R₂ is NO₂, F, Cl, Br, CF₃, CCl₃, or CH₂N(CH₃)₂, and L₁ is a C₂-C₆ alkylene divalent radical or a divalent radical —R₃—X₁—R₄— in which X₁ is an amide linkage of —C(O)NH—, R₃ and R₄ independent of each other are a C₂-C₆ alkylene divalent radical which is optionally substituted with one or more hydroxyl groups.

7. The water-soluble hydrophilic copolymer of claim 6, wherein in formula (I) the boronic acid group of *—B(OH)₂ is at para position.

8. The water-soluble hydrophilic copolymer of claim 6, wherein in formula (I) L₁ is a divalent radical —R₃—X₁—R₄— in which X₁ is an amide linkage of —C(O)NH—, R₃ and R₄ independent of each other are a C₂-C₆ alkylene divalent radical which is optionally substituted with one or more hydroxyl groups.

9. The water-soluble hydrophilic copolymer of claim 6, wherein in formula (I) L₁ is a C₂-C₆ alkylene divalent radical.

10. The water-soluble hydrophilic copolymer of claim 6, wherein in formula (I) R₂ is Cl.

11. The water-soluble hydrophilic copolymer of claim 6, wherein in formula (I) R₂ is NO₂.

12. The water-soluble hydrophilic copolymer of claim 6, wherein the arylborono-containing acrylamido monomer is 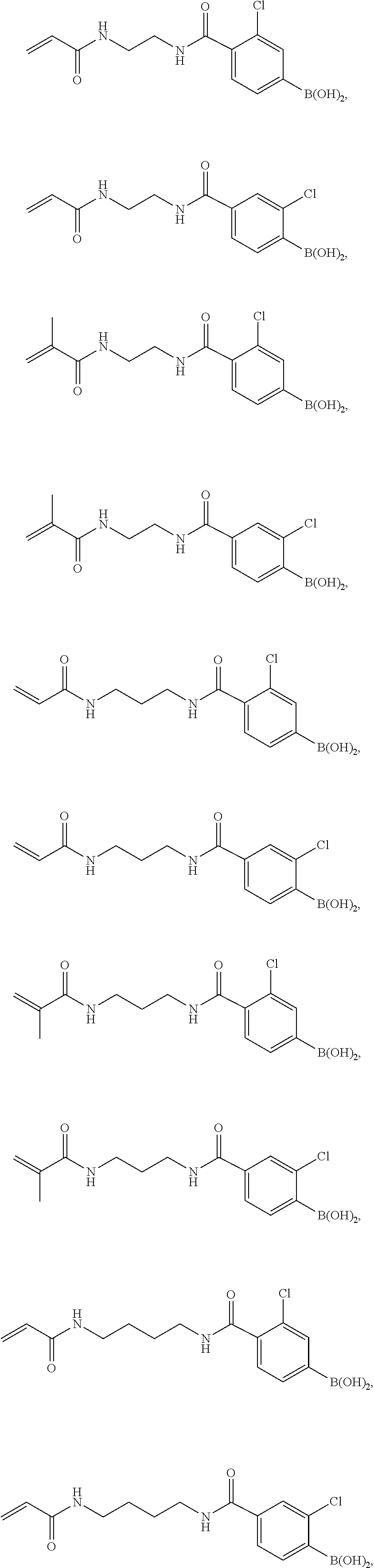
or combinations thereof.

13. The water-soluble hydrophilic copolymer of claim 6, wherein the arylborono-containing acrylamido monomer is 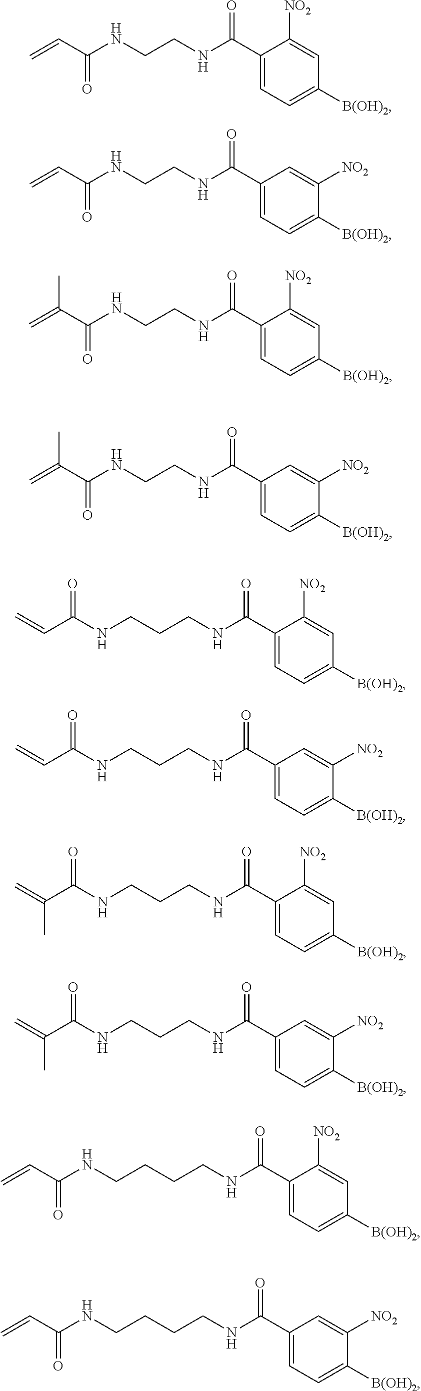
or combinations thereof.

14. The water-soluble hydrophilic copolymer of claim 1, wherein said at least one hydrophilic vinylic monomer comprises (meth)acrylamide, N,N-dimethyl (meth)acrylamide, N-ethyl (meth)acrylamide, N-propyl (meth)acrylamide, N-isopropyl (meth)acrylamide, N-2-hydroxylethyl (meth)acrylamide, N,N-bis(hydroxyethyl) (meth)acrylamide, N-3-hydroxypropyl (meth)acrylamide, N-2-hydroxypropyl (meth)acrylamide, or combinations thereof.

15. The water-soluble hydrophilic copolymer of claim 1, wherein said at least one hydrophilic vinylic monomer comprises 1-methyl-3-methylene-2-pyrrolidone, 1-ethyl-3-methylene-2-pyrrolidone, 1-methyl-5-methylene-2-pyrrolidone, 1-ethyl-5-methylene-2-pyrrolidone, 5-methyl-3-methylene-2-pyrrolidone, 5-ethyl-3-methylene-2-pyrrolidone, 1-n-propyl-3-methylene-2-pyrrolidone, 1-n-propyl-5-methylene-2-pyrrolidone, 1-isopropyl-3-methylene-2-pyrrolidone, 1-isopropyl-5-methylene-2-pyrrolidone, 1-n-butyl-3-methylene-2-pyrrolidone, 1-tert-butyl-3-methylene-2-pyrrolidone, allyl alcohol, or combinations thereof.

16. The water-soluble hydrophilic copolymer of claim 1, wherein said at least one phosphorylcholine-containing vinylic monomer comprises (meth)acryloyloxyethyl phosphorylcholine, (meth)acryloyloxypropyl phosphorylcholine, 4-(meth) acryloyloxy)butyl-2′-(trimethylammonio)ethylphosphate, 2-[(meth)acryloylamino]-ethyl-2′-(trimethylammonio)-ethylphosphate, 3-[(meth)acryloylamino]propyl-2′-(trimethyl-ammonio)ethylphosphate, 4-[(meth)acryloylamino]butyl-2′-(trimethylammonio)ethyl-phosphate, 5-((meth)acryloyloxy)pentyl-2′-(trimethylammonio)ethyl phosphate, 6-((meth)acryloyloxy)hexyl-2′-(trimethylammonio)-ethylphosphate, 2-((meth)acryloyloxy)ethyl-2′-(triethylammonio)ethylphosphate, 2-((meth)acryloyloxy)ethyl-2′-(tripropylammonio)ethyl-phosphate, 2-((meth)acryloyloxy)ethyl-2′-(tributylammonio)ethyl phosphate, 2-((meth)acryloyloxy)propyl-2′-(trimethylammonio)-ethylphosphate, 2-((meth)acryloyloxy)-butyl-2′-(trimethylammonio)ethylphosphate, 2-((meth)acryloyloxy)pentyl-2′-(trimethylammonio)ethylphosphate, 2-((meth)acryloyloxy)hexyl-2′-(trimethylammonio)ethyl phosphate, 2-(vinyloxy)ethyl-2′-(trimethylammonio)ethylphosphate, 2-(allyloxy)ethyl-2′-(trimethylammonio)ethylphosphate, 2-(vinyloxycarbonyl)ethyl-2′-(trimethylammonio)ethyl phosphate, 2-(allyloxycarbonyl)ethyl-2′-(trimethylammonio)-ethylphosphate, 2-(vinylcarbonylamino)ethyl-2′-(trimethylammonio)ethylphosphate, 2-(allyloxycarbonylamino)-ethyl-2′-(trimethylammonio)ethyl phosphate, 2-(butenoyloxy)ethyl-2′-(trimethylammonio)-ethylphosphate, or combinations thereof.

17. The water-soluble hydrophilic copolymer of claim 1, wherein said at least one hydrophilic vinylic crosslinker comprises more preferably selected from the group consisting of N,N-di(meth)acryloyl-N-methylamine, N,N-di(meth)acryloyl-N-ethylamine, N,N′-methylene bis(meth)acrylamide, N,N′-ethylene bis(meth)acrylamide, N,N′-dihydroxyethylene bis(meth)acrylamide, N,N′-propylene bis(meth)acrylamide, N,N′-2-hydroxypropylene bis(meth)acrylamide, N,N′-2,3-dihydroxybutylene bis(meth)acrylamide, combinations thereof.

18. An ophthalmic composition, comprising:
from about 0.1 w/v % to about 10 w/v % of a water-soluble hydrophilic copolymer of claim 1;
one or more buffering agents in an amount sufficient to provide the ophthalmic composition with a pH of from about 6.5 to about 8.2; and
optionally one or more additional excipients and/or one or more additional active ingredients.
